(19) United States
(12) Patent Application Publication
(10) Pub. No.: US 2024/0102023 A1
(43) Pub. Date: Mar. 28, 2024

(54) NOVEL RNA APTAMER INTERVENES ESTROGEN RECEPTOR INTERACTION WITH COACTIVATOR MED1 TO OVERCOME BREAST CANCER METASTASIS

(71) Applicant: University of Cincinnati, Cincinnati, OH (US)

(72) Inventor: Xiaoting Zhang, Mason, OH (US)

(73) Assignee: University of Cincinnati, Cincinnati, OH (US)

(21) Appl. No.: 17/768,023

(22) PCT Filed: Oct. 14, 2020
(86) PCT No.: PCT/US2020/055476

§ 371 Date: Apr. 11, 2022

Related U.S. Application Data
(60) Provisional application No. 62/914,719, filed on Oct. 14, 2019.

(51) Int. Cl. C12N 15/115; A61P 35/00

(52) U.S. Cl. CPC C12N 15/115; A61P 35/00; C12N 2310/335; C12N 2310/3517

(57) ABSTRACT

The present disclosure concerns RNA aptamers that specifically inhibit the protein-protein interaction between MED1 and an estrogen receptor. As shown herein, the aptamers are specific for the LXXLL recognition motif between MED 1 and an estrogen receptor. The present disclosure further concerns pRNA nanoparticles of the RNA aptamers coupled with a further HER2 aptamer that show HER2 specific uptake and subsequent inhibition of cellular proliferation and metastasis in vitro and in vivo. Furthermore, the pRNA nanoparticles showed no adverse effects, signifying a safe and effective composition to specifically target and control HER2 expressing cells.

### RELATED APPLICATION

This application claims priority to U.S. Provisional Patent Application 62/914,719, filed Oct. 14, 2019, the entire contents of which are hereby incorporated by reference.

### STATEMENT REGARDING FEDERALLY SPONSORED RESEARCH AND DEVELOPMENT

This invention was made with Government support under W81XWH-11-1-0018 awarded by the U.S. Department of Defense. The Government has certain rights in the invention.

### FIELD

The present description relates to RNA aptamers with high specificity to disrupt MED1 interaction with estrogen receptors and nanoparticles including such aptamers for enhanced and targeted delivery.

### BACKGROUND

Breast cancer is both the most frequently diagnosed cancer and the leading cause of cancer-related deaths among women in the U.S. and globally (Bray et al. CA: a cancer journal for clinicians. 2018; 68(6):394-424). Metastatic breast cancer (MBC) is currently incurable and has a devastating estimated 5-year survival rate of less than 30% (O'Shaughnessy. The oncologist. 2005; 10(Supplement 3):20-9). While treatments have been developed to improve the prognosis of metastatic breast cancer patients, these are mostly palliative (Reed et al. BMJ supportive & palliative care. 2015; 5(4):358-65).

About 75% of all breast cancers express Estrogen Receptor (ER) a, a nuclear hormone receptor and ligand-dependent transcription factor that has been established as a key contributor to breast oncogenesis (McKenna et al. Endocrine reviews. 1999; 20(3):321-44; Kumar et al. Cell. 1987; 51(6):941-51; Bick et al. Estrogen Receptor-Mediated Gene Transcription and Cistrome. In: Zhang X, editor. Estrogen Receptor and Breast Cancer. Cincinnati: Springer Nature Switzerland AG; 2019. p. 49-70; Jensen. Recent Progr Hormone Res. 1962; 18:387-4142-5). ER-targeting therapies such as selective estrogen receptor modulators (SERMs) and selective estrogen receptor degraders (SERDs) have been developed and widely used for breast cancer treatment (Abderrahman et al. Estrogen Receptor and Breast Cancer: Springer. p. 189-213; Jordan. J. Med. Chem. 2003; 46(6):883-908; Patel et al. Pharmacology & therapeutics. 2018; 186:1-24; McDonnell et al. Curr. Opin. Pharmacol. 2010; 10(6):620-8). However, nearly half of all patients develop resistance to these therapies and their tumors metastasize, which results in impairment to the proper functioning of vital organs and ultimately patient mortality (Shou et al. Journal of the National Cancer Institute. 2004; 96(12):926-35; Osborne et al. Annual review of medicine. 62:233-47; Martin et al. Cancer invasion and metastasis: molecular and cellular perspective. Madame Curie Bioscience Database [Internet]: Landes Bioscience; 2013; Sledge Jr. Journal of oncology practice. 2016; 12(1):6-10).

Multiple studies have implicated the overexpression and/or gene amplification of receptor tyrosine kinase HER2 with increased risk of resistance and metastases in breast cancers (Slamon et al. New England Journal of Medicine. 2001; 344(11):783-92; Eccles. Journal of mammary gland biology and neoplasia. 2001; 6(4):393-406; Yu et al. Oncogene. 2000; 19(53):6115; Slamon et al. Science. 1989; 244(4905):707-12). More recently, research has identified key roles for a tissue-specific ER transcriptional coactivator known as Mediator Subunit 1 (MED1) and its interactions with ER in HER2-mediated tumor onset, growth and metastasis (Yang et al. Cancer research. 2018; 78(2):422-35).

During recent years, RNA has gained recognition as a highly versatile biomaterial with advantageous structure, function and thermodynamic stability, etc. (Hague et al. Wiley Interdisciplinary Reviews: RNA. 2018; 9(1):e1452). The diverse folding patterns of RNAs allow them to form a variety of secondary and tertiary structures, and thus interact with various substrates (Hague et al. Wiley Interdisciplinary Reviews: RNA. 2018; 9(1):e1452; Brion et al. Annual review of biophysics and biomolecular structure. 1997; 26(1):113-37). The RNA aptamer is one type of non-coding RNA that binds desired target proteins, cells, tissues, etc., with high affinity and selectivity (Germer et al. Science. 2000; 287(5454):820-5; Hermann et al. Science. 2000; 287(5454):820-5). RNA aptamers are often selected through a highly characterized in vitro method known as the systematic evolution of ligands by exponential enrichment (SELEX) (Germer et al. Science. 2000; 287(5454):820-5; Hermann et al. Science. 2000; 287(5454):820-5; Guo. Nature nanotechnology. 2010; 5(12):833; Keefe et al. Nature reviews Drug discovery. 2010; 9(7):537; Shu et al. Nature protocols. 2013; 8(9):1635). However, a common obstacle of RNA aptamers and other small RNA-based therapies remains to be their systemic delivery. Thus, there is a need for a successful delivery system of nanoparticles in vivo that are capable of specific targeting to HER2 cells to disrupt the aberrant signaling.

### SUMMARY OF THE INVENTION

The present description concerns a Mediator Subunit 1 (MED1)-estrogen receptor (ER) binding inhibitor with a ribonucleic acid (RNA) sequence having at least 70% identity to a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10. In some aspects, the nucleic acid sequence has at least 85% identity. In further aspects, the nucleic acid sequence includes 20 or more consecutive bases to the sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

In certain aspects, the MED1-ER binding inhibitor RNA sequence includes SEQ ID NO: 9 and/or SEQ ID NO: 10.

In some aspects, one or more nucleic acids of the RNA sequences include a modification that confers nuclease resistance. In certain aspects, such modification may include labelling a base with a fluorine or an O-methyl. In certain aspects, all uracil residues may be 2′ fluoro-labeled. In even further aspects, all cytosine residues may be 2′ fluoro-labeled.

The present disclosure also includes a pRNA nanoparticle of at least one of the RNA aptamer sequences and a 3-way junction (3WJ) nucleotide sequence. The 3WJ nucleotide sequence may include the combination of SEQ ID NO: 13, SEQ ID NO: 14 and SEQ ID NO: 15.

In further aspects, the pRNA nanoparticle may include an additional aptamer, including a HER2 aptamer as set forth in SEQ ID NO: 18 or SEQ ID NO: 25.

In certain aspects, the pRNA includes nucleic sequences having at least 85% identity to the sequences as set forth in SEQ ID NOS: 19, 20 and 21. In other aspects, the pRNA includes a nucleic acid having at least 85% identity to the sequence as set forth in SEQ ID NO: 26.

In some aspects, the pRNA may include a modification to confer nuclease resistance, such as by labeling a base with a fluorine or an O-methyl. In certain aspects, all uracil residues are 2′ fluoro-labeled and/or all cytosine residues are 2′ fluoro-labeled.

The present disclosure further includes methods of inhibiting MED1 interacting with an estrogen receptor by administering the RNA aptamers and/or pRNA nanoparticles as set forth herein. In some aspects, the methods may include administering to a cell, in vitro or in vivo, a composition of an RNA aptamer having at least 85% identity to a nucleic acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

In some aspects, the composition further includes a pharmaceutically acceptable carrier, a diluent and/or an excipient.

In aspects where the cell is in vivo, the composition may be administered systemically.

The present disclosure further includes methods for treating a HER2 associated tumor. In some aspects, the tumor is within a subject. The methods may include systemically administering a composition comprised of a pRNA nanoparticle. In some aspects, the pRNA nanoparticle is a self-assembled structure of 3 sequences having at least 85% identity to SEQ ID NOS: 19, 20 and 21. In other aspects, the pRNA is a self-assembled structure from a sequence having at least 85% identity to SEQ ID NO: 26. In some aspects, the composition may include a pharmaceutically acceptable carrier, a diluent and/or an excipient. The composition may be administered systemically.

The HER2 associated tumor may be one or more of a breast tumor, a bladder tumor, a gastric tumor, a gallbladder tumor, a hepatic tumor, a cervical tumor, a uterine tumor or a testicular tumor.

In some aspects, the method may include co-administering to the subject the composition with a further agent, such as a chemotherapeutic. In certain aspects, the further chemotherapeutic may be selected from raloxifene, tamoxifen, abemacicib, paclitaxel, everolimus, ado-trastuzumab emtansine, alpelisib, anastrozole, pamidronate, exemestane, atezolizumab, capecitabine, cyclophosphamide, docetaxel, doxorubicin, epirubicin, fluorouracil, fam-trastuzumab deruxtecan-nxki, toremifene, fulvestrant, letrozole, gemcitabine, goserelin, trastuzumab, palbociclib, ixabepilone, ribociclib, lapatinib ditosylate, letrozole, olaparib, megestrol, methotrexate, neratinib, pertuzumab, sacituzumab govitecan-hziy, thiotepa, tucatinib, vinblastine, or combinations thereof.

In further aspects, the RNA aptamers can be defined by a secondary and/or tertiary structure. As set forth herein, RNA aptamers to disrupt MED1-ER binding inhibitor may include a ribonucleic acid (RNA) aptamer structure with a particular order to allow for self-assembly of a desired secondary and/or tertiary structure. In some aspects the bases are arranged in an order to provide a first stem of 5 base pairs matched with 5 bases of the opposing end of the sequence; followed by 8 unmatched base pairs; followed by a first stem and loop portion of 5 matched pairs and 8 unmatched base pairs; followed by 2 unmatched base pairs; followed by a second stem and loop portion of 4 matched base pairs and 6 unmatched bases; followed by one unmatched base; and terminating with the 5 bases of the opposing end of the first stem. In further aspects, a pRNA nanoparticle of the RNA structure and a 3-WJ RNA sequence is included.

### BRIEF DESCRIPTION OF THE DRAWINGS

FIG. 1 shows isolation of MED1 LXXLL (SEQ ID NO: 35) Motif-targeting RNA Aptamers through SELEX. FIG. 1A shows a schematic of the SELEX procedure used to isolate MED1 LXXLL (SEQ ID NO: 35) motif-selective RNA aptamers using FIG. 1B purified starting RNA library generated by T7 RNA transcription of a random DNA pool, and FIG. 1C purified MED1 WT (LXXLL (SEQ ID NO: 35)) and MED1 Mutant (LXXAA (SEQ ID NO: 36)) proteins. FIG. 1D shows double stranded DNA (dsDNA) generated following PCR amplification and reverse transcription. FIG. 1E shows the RNA recovery rate after each round of SELEX. FIG. 1F shows the sequences of the top 8 most enriched RNA aptamer candidates.

FIG. 2 shows MED1SP RNA Aptamer Disrupts ER/MED1 Interaction, Breast Cancer Cell Growth, Migration and Invasions in Vitro. FIG. 2A shows a schematic of the predicted folded structure of MED1SP RNA aptamer by mFOLD. FIG. 2B shows ERE-luciferase reporter assays of BT-474 cells transfected with 10 μg/mL of control Scramble, full length P or MED1SP aptamer. FIG. 2C shows GST Pull-down assays using purified GST-ER and MED1 WT in the presence of control or increasing concentrations of MED1SP. FIG. 2D shows GST Pull-down assays using 2 μg of MED1SP and nuclear extract (NE), followed by Western blot analyses of indicated proteins. FIG. 2E shows BT-474 cells treated with 10 μg/mL control or MED1SP for 48 hours were assayed for cell proliferation via MTT assay. FIG. 2F and FIG. 2G shows cells treated as in FIG. 2(E) were seeded for transwell (FIG. 2F) migration and (FIG. 2G) invasion assays with quantifications shown respectively. The scale bar presented represents 50 μm.

FIG. 3 shows generation and Characterization of 3-WJ pRNA-HER2-MED1SP Nanoparticles for Specific Delivery into Breast Cancer Cells. FIG. 3A shows a schematic of p-HER2-MED1SP following incorporation of SP and HER2 B3 RNA aptamer into 3-WJ motif scaffold. FIG. 3B shows equal molar amounts of P1, P2, and P3 strands were annealed and analyzed by 8% Native PAGE gel electrophoresis. FIG. 3C shows Atomic Force Microscopy (AFM) analyses of annealed p-HER2-MED1 SP nanoparticles. FIG. 3D and FIG. 3E shows stability of annealed nanoparticles and unmodified RNA was measured by 8% Native PAGE gel electrophoresis after exposure to FIG. 3D DMEM+10% FBS or FIG. 3E RNase A. FIG. 3F shows confocal microscopy analyses of nanoparticle uptake by BT-474 cells treated with control, p-HER2-Scramble, and p-HER2-MED1SP. Scale Bar: 10 μm.

FIG. 4 shows p-HER2-MED1SP RNA Nanoparticles Disrupt Cell Growth, ER Target Gene Expression and Metastatic Capabilities of HER2-expressing Breast Cancer Cells in Vitro. FIG. 4A shows BT-474 breast cancer cells were treated with 10 μg/mL of p-HER2-Scramble or p-HER2-MED1SP for 48 hours and cell growth was assessed by MTT assay. FIG. 4B shows estrogen (E2)-starved BT-474 cells were treated with 10 μg/mL of p-HER2-Scramble or p-HER2-MED1SP nanoparticles overnight, and ERE-luciferase reporter gene expression was measured. FIG. 4C and FIG. 4D show BT-474 cells treated as in FIG. 4(A) were seeded and measured for FIG. 4C migration and FIG. 4D invasion capabilities via transwell assays and quantified. Scale Bar: 50 μm FIG. 4E, FIG. 4F and FIG. 4G show mRNA levels of endogenous ER-target genes TFF-1 (FIG. 4E), c-Myc (FIG. 4F), and cyclin D1 (FIG. 4G) were measured using realtime PCR following 48 hour treatment with p-HER2-Scramble or p-HER2-MED1SP.

FIG. 5 shows p-HER2-MED1SP Nanoparticles Inhibit Breast Cancer Tumor Growth and Metastasis in Vivo. FIG. 5A shows BT-474 orthotopic xenograft mouse models were treated with 4 mg/kg of Alexa647-tagged p-HER2-Scramble or Alexa647-tagged p-HER2-MED1SP nanoparticles via tail vein injection once a week for 3 weeks and tumor growth was measured with calipers. FIG. 5B shows that following the final week of tumor measurement (week 4), organs and tumors were harvested from sacrificed mice and tumors were weighed. FIG. 5C shows heart, spleen, lung, kidneys and liver were imaged via IVIS Lumina imaging system. FIG. 5D shows sections from fixed and embedded whole lung tissues were stained with H&E and the numbers of metastatic lung nodules were counted. Scale bar: 100 μm. FIG. 5E shows tumors were fixed, embedded and sectioned and the expression of Ki-67 was measured by IHC staining. Scale bar: 50 μm. FIG. 5F, FIG. 5G and FIG. 5H show total RNA was extracted from tumors using TRIZOL and real-time PCR was used to measure the expression of TFF-1 (FIG. 5F), c-Myc (FIG. 5G), and cyclin D1 (FIG. 5H).

FIG. 6 shows functional Analyses of the Top 8 Enriched RNA Aptamer Candidates. FIG. 6A shows the binding of these RNA aptamer candidates and control Scramble (S) aptamer to immobilized MED1 WT (LXXLL (SEQ ID NO: 35)) protein or MED1 Mutant (LXXAA (SEQ ID NO: 36)) protein were examined. FIG. 6B shows RNA aptamers were tested for their ability to block the ER/MED1 interaction via GST pulldown assays. FIG. 6C shows estrogen (E2)-starved MCF-7 breast cancer cells were transfected with 10 μg/mL of indicated RNA aptamer and ERE-luciferase reporter activities were measured. FIG. 6D shows MCF-7 cells were treated with 10 μg/mL of indicated RNA aptamer for 48 hours and cell growth was measured by MTT assay. FIGS. 6E and 6F show cells treated as in FIG. 6D and migration capabilities were measured via transwell assay (FIG. 6E) and quantified (FIG. 6F).

FIG. 7 shows uptake and functional analyses of p-HER2-MED1SP Nanoparticles using MCF-7/HER2 Breast Cancer Cells. FIG. 7A shows MCF-7/HER2 breast cancer cells treated with PBS, 10 μg/mL of Alexa647-tagged p-HER2-Scramble or Alexa647-tagged p-HER2-MED1SP nanoparticles and analyzed by confocal microscopy. Scale bar: 10 μm. FIG. 7B shows MCF-7/HER2 breast cancer cells treated as in FIG. 7A for 48 hours and cell growth was measured by MTT assay. FIG. 7C shows estrogen-starved MCF-7/HER2 breast cancer cells treated as in (A) overnight and EREluciferase reporter gene expression was measured as indicated. FIG. 7D, FIG. 7E and FIG. 7F show MCF-7/HER2 cells treated as in (B) and the expression of endogenous ER-target genes TFF-1 (FIG. 7D), c-Myc (FIG. 7E), and cyclin D1 (FIG. 7F) were measured by real-time PCR analysis.

FIG. 8 shows p-HER2-MED1SP Nanoparticle Treatment Does Not Affect Body Weight or Histology of Vital Organs. FIG. 8A shows body weight of BT-474 orthotopic xenograft mice treated with 4 mg/kg p-HER2-Scramble or p-HER2-MED1SP nanoparticles by tail vein injection once a week for 3 weeks measured at indicated times. FIG. 8B shows organs harvested after completion of nanoparticle treatment that were fixed, embedded, sectioned and stained with H&E for morphology analyses.

FIG. 9 shows p-HER2-MED1SP Nanoparticles Do Not Induce Apoptosis of Xenografted Tumors. FIG. 9A shows tumors from p-HER2-Scramble and p-HER2-MED1SP treated mice were harvested, fixed, embedded, sectioned and stained for cleaved Caspase-3 (FIG. 9A) and quantified (FIG. 9B). Scale bar: 50 μm.

### DETAILED DESCRIPTION

The present disclosure concerns aptamers of ribonucleic acid (RNA) nucleic acid residues that, as disclosed herein, show selectivity for inhibiting the coactivator MED1 from interacting and/or activating an estrogen receptor. In some aspects, the RNA aptamers interfere, block or inhibit at least one LXXLL (SEQ ID NO: 35) motif of MED1 from interacting, binding and/or activating and/or stimulating an estrogen receptor (ER), such as ERα.

MED1 belongs to a subpopulation of TRAP/Mediator complexes that interact with the AF-2 domain of ERα directly through its two LXXLL (SEQ ID NO: 35) motifs (Zhang et al. Molecular cell. 2005; 19(1):89-100; Kornberg. Trends in biochemical sciences. 2005; 30(5):235-9; Allen et al. Nature reviews Molecular cell biology. 2015; 16(3):155; Leonard et al. Journal of Zhejiang University-SCIENCE B. 2019; 20(5):381-90; Malik et al. Nat Rev Genet. 2010; 11(11):761-72. Epub 2010/10/14. doi: nrg2901 [pii]10.1038/nrg2901. PubMed PMID: 20940737). Interestingly, mutation of the MED1 LXXLL (SEQ ID NO: 35) motifs to LXXAA (SEQ ID NO: 36) in a MED1 mutant knock-in mouse model generated viable, fertile, and grossly normal mice with defects only apparent in pubertal mammary gland development (Jiang et al. PNAS. 2010; 107(15):6765-70). Further, while estrogen-induced mammary duct growth was inhibited in vivo in these mice, estrogen-stimulated weight gain of other estrogen-responsive tissue such as uteri was unaffected (Jiang et al. PNAS. 2010; 107(15):6765-70). Moreover, recent studies have shown that the MED1 LXXLL (SEQ ID NO: 35) motifs and their interactions with ERs play a pivotal role in HER2-mediated breast cancer growth and metastasis (Yang et al. Cancer research. 2018; 78(2):422-35). Thus, when the MED1 mutant knock-in mouse was crossed with an MMTV-HER2 transgenic mouse, the progeny exhibited delayed tumor onset, slowed tumor growth, a loss in lung metastasis in addition to lowered cell proliferation, angiogenesis, and cancer stem cell formation (Yang et al. Cancer research. 2018; 78(2):422-35). These findings suggested the targeting of MED1 LXXLL (SEQ ID NO: 35) motifs for tissue- and gene-specific disruption of ER signaling (Leonard et al. Journal of Zhejiang University-SCIENCE B. 2019; 20(5):381-90; Malik et al. Nat Rev Genet. 2010; 11(11):761-72. Epub 2010/10/14. doi: nrg2901 [pii]10.1038/nrg2901. PubMed PMID: 20940737; Acevedo et al. Molecular cell. 2004; 13(5):725-38). Notably, MED1 is overexpressed in about half of all breast cancers and co-amplifies with HER2 in nearly all instances (Zhu et al. PNAS. 1999; 96(19):10848-53; Leonard et al. Estrogen Receptor and Breast Cancer: Springer; 2019. p. 379-403).

In this the present disclosure, novel MED1 LXXLL (SEQ ID NO: 35)-motif targeting RNA aptamers have been identified through a SELEX approach to disrupt the interaction between ER and its key tissue-specific coactivator MED1. As set forth herein, it is also confirmed that the MED1 RNA aptamers, including MED1SP, can inhibit ER-mediated reporter and endogenous gene expression, breast cancer cell growth and migration and invasion capabilities in vitro. As further described herein, coupling the MED1 aptamers with a HER2 aptamer can in some aspects provide cell specific inhibition of ER activity in vitro and in vivo.

As is also described herein, the RNA aptamers are specific for targeting the protein-protein interaction (PPI) between MED1 and the estrogen receptor. As is further described herein, the RNA aptamers specifically target the LXXLL (SEQ ID NO: 35) motif MED1 uses to interact and/or activate an estrogen receptor. The present disclosure sets forth herein isolated RNA aptamers that are capable of targeting and disrupting an important PPI between the key breast cancer driver ER and its coactivator MED1. Although LXXLL (SEQ ID NO: 35) motifs are present in a variety of ER coactivators, it is also known that the coactivator-specific sequences flanking the LXXLL (SEQ ID NO: 35) motif are important for their interactions with ER (Savkur et al. J Pept Res. 2004; 63(3):207-12. PubMed PMID: 15049832, Coulthard et al. Journal of Biological Chemistry. 2003; 278(13):10942-51). This has led to the concept of potentially targeting LXXLL (SEQ ID NO: 35) motifs to inhibit particular ER-coactivator interactions for the tissue- and gene-specific disruption of ER signaling (Leonard et al. Journal of Zhejiang University-SCIENCE B. 2019; 20(5):381-90, Jiang et al. PNAS. 2010; 107(15):6765-70). It is a further facet of the present disclosure that the RNA aptamers are specific for the MED1 LXXLL (SEQ ID NO: 35) motif in vitro and in vivo.

In further aspects, the present disclosure also concerns a pRNA nanoparticle that contains or carries the MED1 aptamers as described herein. Construction and systemic administration of pRNA-HER2-MED1SP RNA nanoparticles in vivo in human breast cancer orthotopic xenograft mouse models as described herein demonstrates the RNA nanoparticles specifically target HER2-expressing tumors and markedly block their growth and lung metastasis without apparent adverse effects. Relatedly, the pRNA nanoparticles are generally understood to have excellent biosafety and pharmacodynamic properties with little toxicity in a growing number of disease models (Jasinski et al. ACS nano. 2017; 11(2):1142-64), providing advantageous therapeutic regimens for patients with minimized side effects. The results obtained with the RNA aptamers and the pRNA nanoparticles demonstrate no apparent toxicity, body weight change or altered histology of vital organs following systemic treatment.

Significantly, ER/HER2 double positive luminal B breast cancer subtypes are especially difficult to treat because they are highly metastatic and resistant to current therapies. Therefore, it is an important facet of the present disclosure that the p-HER2-MED1SP nanoparticles as set forth herein exhibit great efficacy in inhibiting ER+/HER2+ breast cancer metastasis both in vitro and in vivo.

In addition, HER2 is known to be overexpressed in a variety of other cancer types, including lung, gastric, esophageal, ovarian and endometrial cancers (Iqbal et al. Molecular biology international. 2014; 2014). Further, ER signaling that is driven by both hormone expression and HER2-activation has already been shown to play important roles in a number of these cancers (Thomas et al. Nat Rev Cancer. 2011; 11(8):597-608. Epub 2011/07/23. doi: 10.1038/nrc3093. PubMed PMID: 21779010, Verri et al. Oncology. 2005; 68(2-3):154-61). Accordingly, it is a further aspect of the present invention that the p-HER2-MED1SP nanoparticles and the RNA aptamers have further application beyond breast cancer.

In addition, with respect to ER-driven cancers that do not express HER2, the HER2 aptamer of the pRNA as described herein can be replaced with other targeting aptamers (e.g. EpCAM, EGFR, CD44) or moieties (e.g. folate) to facilitate targeted delivery of the pRNA nanoparticles and further broaden the application of the MED1SP aptamers (Jasinski et al. ACS nano. 2017; 11(2):1142-64).

### RNA Aptamers

RNA aptamers are defined generally as RNA oligonucleotides that bind to a specific target with high affinity and specificity. Aptamers may be chemically synthesized, then selected for a desired binding profile through the well known SELEX process and derivations such as Cell-SELEX and Tissue-SELEX.

Generally, an RNA aptamer may be of from about 56 to about 120 nucleotides in length. An RNA aptamer, in some aspects, may include a variable region and a constant region. The variable region is more centrally-located and may feature a length in a region of from about 20 to about 80 nucleotides. Constant regions may be located on either or both the 5′ and 3′ sides of the variable region and may each independently be of from about 18 to about 20 nucleotides.

In some aspects, the aptamers are single stranded ribonucleic acid sequences as set forth herein. It will however be appreciated that complementary strands and double stranded variants are also included, as well as variants that include additional nucleic acids of about 1 to about 15 bases in length, including 2, 3, 4, 5, 6, 7, 8, 9, 10, 11, 12, 13, and 14 nucleic acids on either or both of the termini of the aptamer (5′ and/or 3′). In further aspects, it is to be understood that single-stranded (ss) and double-stranded (ds) deoxyribonucleic acid (DNA) variants are also considered, as are L- or left-hand variants.

In some aspects, the present disclosure concerns aptamers with a RNA structure that interrupts the ability of MED1 to interact with or activate an estrogen receptor. In certain aspects, the RNA aptamers inhibit the PPI between MED1 and an ER. As set forth herein, the present disclosure has identified several RNA aptamers that bind to the key LXXLL (SEQ ID NO: 35) motif of MED1 to negatively affect its activity with respect to an estrogen receptor. In some aspects, the RNA aptamers further do not bind a LXXAA (SEQ ID NO: 36) motif. In further aspects, the RNA aptamers are specific to the MED1 LXXLL (SEQ ID NO: 35) motif.

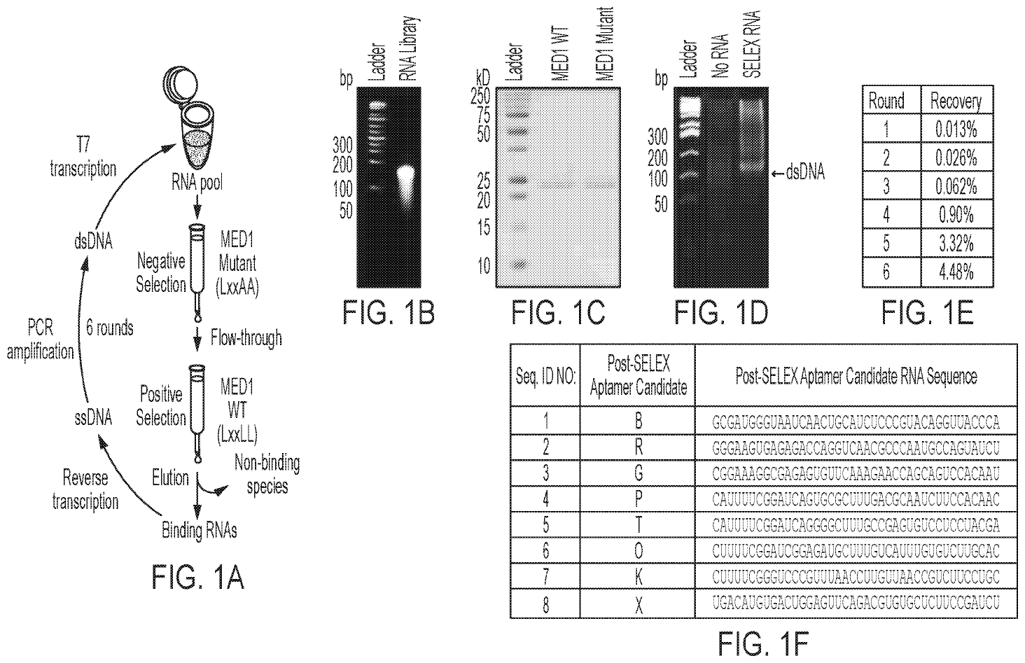

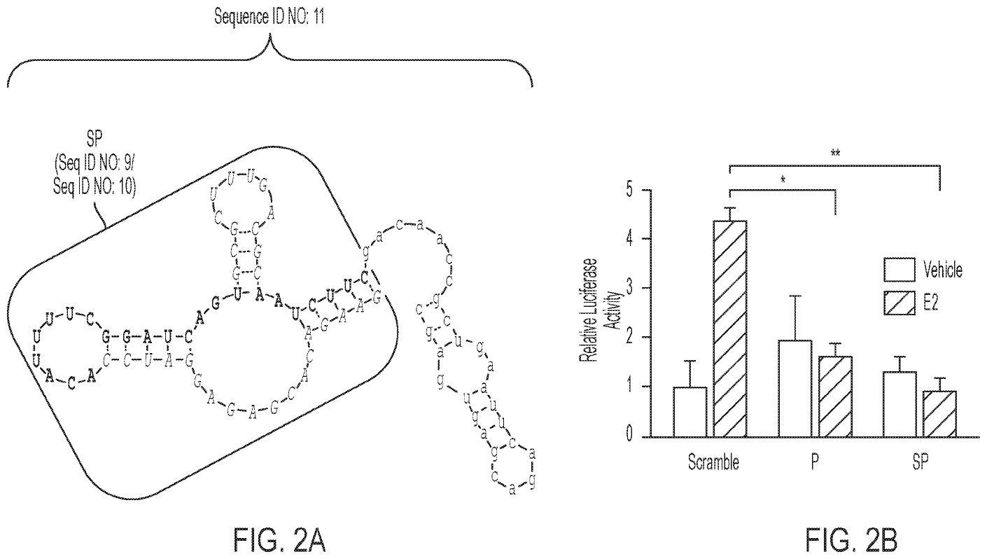

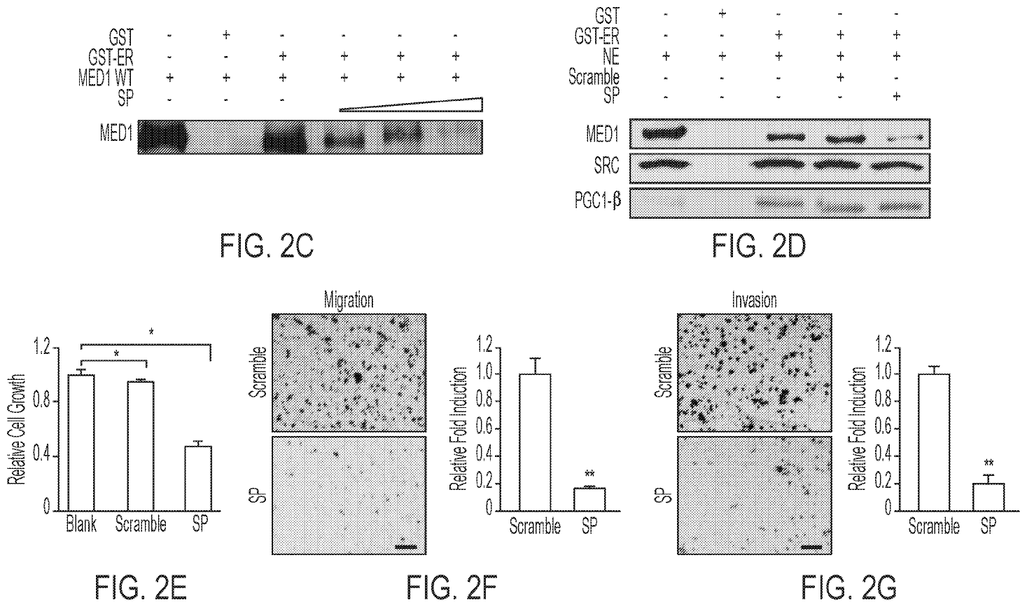

In some aspects, the RNA aptamers of the present disclosure may include one or more of the sequences as set forth in SEQ ID NOS: 1-11 and FIGS. 1 and 2:

(SEQ ID NO: 1; Aptamer B))
GCGAUGGGUAAUCAACUGCAUCUCCCGUACAGGUUACCA
(SEQ ID NO: 2; Aptamer R)
CGGAAGUGAGAGACCAGGUCAACGCCCAAUGCCAGUAUCU
(SEQ ID NO: 3; Aptamer G)
CGGAAAGGCGAGAGUGUUCAAAGAACCAGCAGUCCACAAU
(SEQ ID NO: 4; Aptamer P)
CAUUUUCGGAUCAGUGCGCUUUGACGCAAUCUUCCACAAC
(SEQ ID NO: 5; Aptamer T)
CAUUUUCGGAUCAGGGGCUUUGCCGAGUGUCCUCCUACGA
(SEQ ID NO: 6; Aptamer O)
CUUUUCGGAUGGAGAUGCUUUGUCAUUUGUGUCUUGCAC
(SEQ ID NO: 7; Aptamer K)
CUUUUCGGGUCCCGUUUAACCUUGUUAACCGUCUUCCUGC
(SEQ ID NO: 8; Aptamer X)
UGACAUGUGACUGGAGUUCAGACGUGUGCUCUUCCGAUCU
(SEQ ID NO: 9); shortened P)
CAUUUUCGGAUCAGUGCGCUUUGACGCAAUCUUC
(SEQ ID NO: 10; full length SP in FIG. 2)
GAAGACACGAGAGGAUCCACAUUUUCGGAUCAGUGCGCUUUGACGCAAU
CUUC
(SEQ ID NO: 11; FIG. 2A)
GAAGACACGAGAGGAUCCACAUUUUCGGAUCAGUGCGCUUUGACGCAAU
CUUCGACAACCGCUGAAUUCAGACGAGUGAGC.

It will be appreciated that the present disclosure further encompasses, in some aspects, nucleic acid sequences, including RNA (including L- and D-variants), ssDNA and dsDNA having at least about 70% identity to SEQ ID NOS: 1-11, including about 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.9 and 100% identity. In certain aspects, the RNA aptamers may include a RNA sequence of about 85 to about 100% identity with the sequences as set forth in SEQ ID NOS: 1-11. In further aspects, the RNA aptamers include a RNA sequence having from about 85 to 100% identity to the sequences as set forth in SEQ ID NOS: 1-11 and wherein the sequence includes from about 20 to about 41 consecutive base sequences from SEQ ID NOS: 1-11, including about 21, 22, 23, 24, 25, 26, 27, 28, 29, 30, 31, 32, 33, 34, 35, 36, 37, 38, 39, and 40 consecutive base sequences.

In further aspects, the RNA aptamers and additional constructs including such aptamers, such as the pRNA nanoparticles described herein, are resistant to nuclease degradation. As set forth in the examples, 2′fluoro labeling of cytosine and uracil was utilized. It will be appreciated that the RNA aptamers can be prepared with any combination of 2′ fluoro-labeling, including cytosine, adenosine, uridine and/or guanosine. It will also be appreciated that the 2′ fluoro-labeling can be replaced or combined with other approaches to providing nuclease resistance, including the incorporation of 2′ O-methyl adenosine, 2′ O-methyl cytosine, 2′ O-methyl guanosine, 2′ O-methyl uridine, 3′ deoxy adenosine (2′-5′ linked), 3′ deoxy cytosine (2′-5′ linked), 3′ deoxy guanosine (2′-5′ linked), 3′ O-methyl adenosine, 3′ O-methyl cytosine, 3′ O-methyl guanosine, 3′ O-methyl uridine, 3′ ribo-adenosine 2′-5′ linked), 3′ ribo-cytosine (2′-5′ linked), 3′ ribo-guanosine (2′-5′ linked), 3′ ribo-uridine (2′-5′ linked), L or left turning bases, L or left turning RNA aptamers, phosphorothioate (SOX) linkers, C-5 propyne cytosine analogs and/or C-5 propyne uridine analogs.

In some aspects, the RNA aptamers were identified by selection from a created library of randomly assembled nucleotides. For a starting library design, constant sequences (SEQ ID NOS: 23 and 24) flanking a randomized region were arranged to prevent hairpin-folding and allow for greater flexibility and diversity. The DNA library featured 41 nucleotide (nt) random sequences flanked by constant sequences (GCACAGCTCGTCTGAATTCTG)-N₄₁-(GTGGATCCTCTCGTGTCTTCCTATAGTGAGTCGTATTA) or SEQ ID NO: 23-N₄₁-SEQ ID NO: 24. RNA transcriptions were then carried out using 2′-fluoro-modified UTPs and CTPs and unmodified ATPs and GTPs using RNA polymerases (Zhang et al. ACS nano. 2017; 11(1):335-46). It will be appreciated that other fluoro-labeled bases could be utilized instead and that fluoro labeling can protect the RNA aptamer from degradation. The resultant RNA was analyzed by a urea denaturing gel, from which it was excised and eluted to provide an RNA library of aptamers.

The SELEX method is known as an approach to identify aptamers of interest for a particular target. In the present disclosure, SELEX was applied to isolate a pool of RNA aptamers that bind to MED1 LXXLL (SEQ ID NO: 35) motifs to disrupt the ER/MED1 interaction (see, e.g., SEQ ID NOS: 1-8) as shown in FIG. 1A. The purified RNA library was loaded onto an immobilized MED1 Mutant (LXXAA (SEQ ID NO: 36)) protein column and the flow-through was then incubated with purified MED1 WT (LXXLL (SEQ ID NO: 35)) protein (FIG. 1C). Any RNA species that bound to MED1 WT but not MED1 mutant proteins were then eluted, reverse transcribed and further amplified by PCR into double stranded DNA (dsDNA) (FIG. 1D). Double-stranded DNA (dsDNA) was then transcribed back into an enriched RNA library and used to begin a further SELEX cycle. Importantly, the recovery rate of RNA was increased significantly during each round of SELEX (FIG. 1E), and the resultant sequences of the top 8 most enriched RNA aptamers after 6 rounds are shown in FIG. 1F (SEQ ID NOS: 1-8).

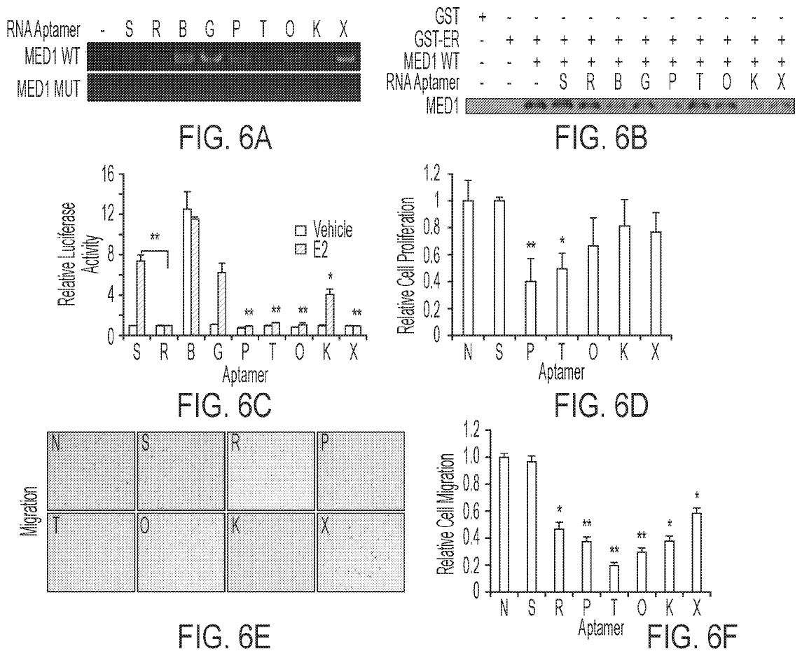

For functional characterization of the identified eight RNA aptamer candidates, basic binding assays were performed to compare binding abilities to MED1. As shown in FIG. 6A, in contrast to the control scramble (S) aptamer, many of these MED1 RNA aptamers (B, G, P, O, K and X, i.e. SEQ ID NOS: 1, 3, 4, 6, and 8) bind to MED1 WT but not MED1 mutant proteins. Further, GST pull-down assays demonstrated the ability of aptamer candidates B, G, P, O, K, and X (i.e. SEQ ID NOS: 1, 3, 4, 6, and 8) to significantly disrupt the interaction between ER and MED1 (FIG. 6B). Moreover, the ability of each individual aptamer to affect ER-mediated gene expression in ER positive MCF-7 breast cancer cells was measured and the results showed that aptamers R, P, T, O, X (i.e. SEQ ID NOS: 2, 4, 5, 6, and 8) were able to strongly inhibit estrogen-dependent ER-reporter gene expression (FIG. 6C). Finally, MTT cell proliferation assays were carried out along with transwell migration assays and it was found that aptamers P and T (SEQ ID NOS: 4 and 5) have the most significant effects on growth (FIG. 6D) while aptamers P, T, and O (SEQ ID NOS: 4, 5, and 6) resulted in the greatest loss in migration capabilities (FIG. 6E, 6F). Based on these evidences, RNA aptamer P was selected for further development as an MED1 aptamer candidate due to demonstrated capabilities in binding MED1 WT, disrupting the ER/MED1 interaction, ER-dependent transcriptional activity, and inhibition of both growth and migration of breast cancer cells.

### RNA Aptamer Modification

Identified RNA aptamers can, in some aspects, be further modified to optimize the tertiary structure. For example, as set forth herein, the P aptamer (SEQ ID NO: 4) was modified to a shortened version. In some aspects, based on the ability to inhibit ER-dependent transcription, breast cancer cell growth and metastatic capabilities, RNA aptamer P (SEQ ID NO: 4) was identified and shortened to a further functional form MED1SP (SEQ ID NO: 9). Upon further analysis of the predicted secondary structure of the P aptamer provided by mFOLD, a loop-stem-loop-stem-loop motif was identified (FIG. 2A) (CAUUUUCGGAUCAGUGCGCUUUGACGCAAUCUUC (SEQ ID NO: 9); shortened P) and GAAGACACGAGAGGAUCCACAUUUUCGGAUCAGUGCGCUUUGACGCAAUCUUC (SEQ ID NO: 10; full length SP within box of FIG. 2A).

Deletion-mapping experiments were conducted and identified that the shortened P (SP) aptamer (MED1SP) can block the expression of ER-reporter genes as efficiently as the full-length P aptamer (FIG. 2B). Further, MED1SP can effectively block the ER/MED1 interaction in GST pull-down assays (FIG. 2C).

It is also an aspect of the present disclosure that the RNA aptamers are site specific to the MED1 LXXLL (SEQ ID NO: 35) motif. While the interaction of ER/MED1 is disrupted by MED1SP, it was confirmed that other transcriptional ER co-activators like SRC and PGC-1(3, both of which contain LXXLL (SEQ ID NO: 35) motifs, still bind to an ER (FIG. 2D). Moreover, MTT assays confirmed that MED1SP significantly inhibits cell growth (FIG. 2E). Further, transfection of MED1SP into BT-474 cells, a HER2-overexpressing metastatic breast cancer cell line, also resulted in a loss in their migration and invasion capabilities, as demonstrated by transwell migration and invasion assays (FIG. 2F, 2G). Overall, these data support a functional selectivity of the MED1SP RNA aptamer and its ability to inhibit ER-mediated transcription and functions.

In other aspects, it will be appreciated that the tertiary structure of the SP aptamer can be achieved with different base pairings. The tertiary structure as set forth in FIG. 2A is determined by the sequence of an initial stem of 5 matched base pairs, followed by 8 unmatched base pairs that lead to a stem and loop of 5 matched pairs in the stem and 8 unmatched base pairs in between to form the loop. The structure continues with 2 unmatched base pairs leading to a second stem-loop of 4 matched base pairs for the stem and 6 unmatched bases in the loop, with one unmatched base then leading back to the initial stem of the 5 matched base pairs to complete the structure. Accordingly, in some aspects, additional RNA aptamers are encompassed by the present disclosure that are of a similar length with the same criteria of matching and unmatching bases to provide the structure as set forth in FIG. 2A.

pRNA Delivery Systems

The present disclosure also concerns pRNA nanoparticles that feature the MED1 targeting RNA aptamers. In some aspects, the pRNA nanoparticle also feature 2′-fluoro labeling. The pRNA nanoparticles provide an ultra-compact and highly stable three-way junction (3-WJ) pRNA nanoparticle delivery systems.

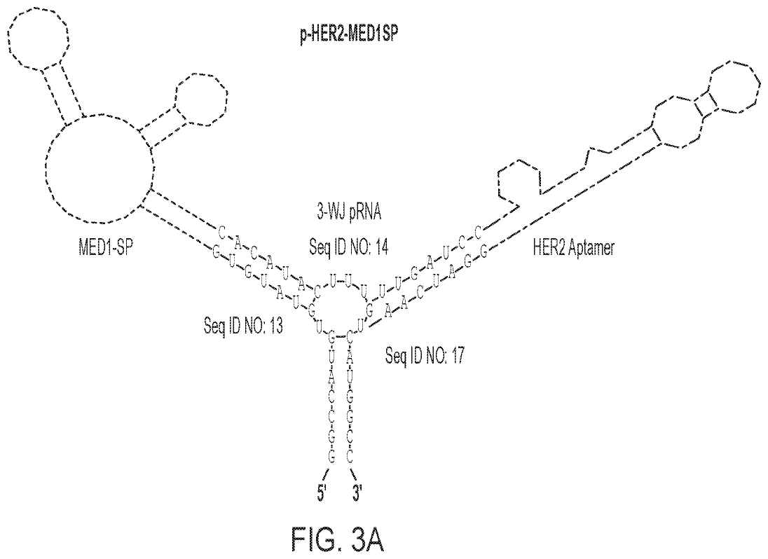

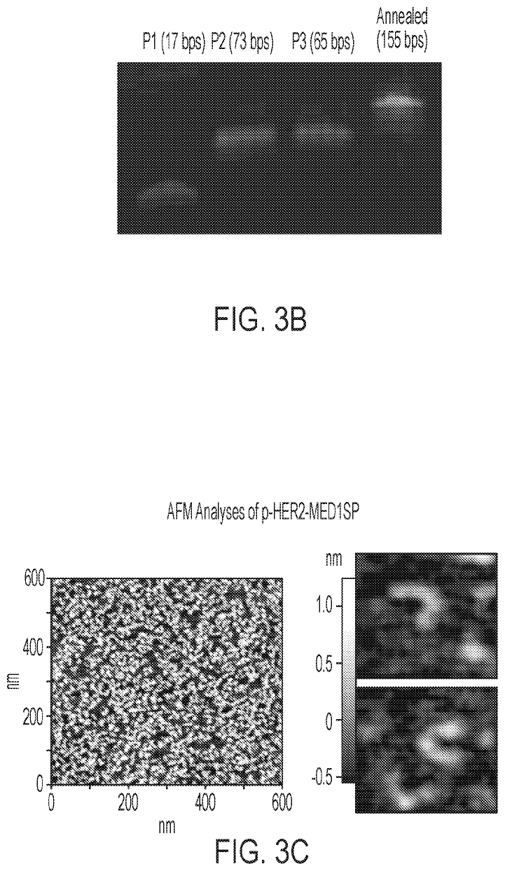

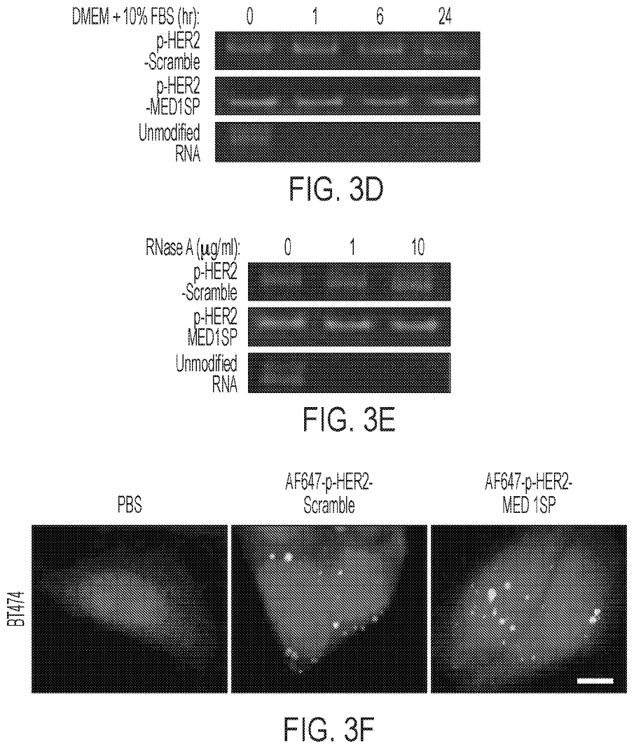

In some aspects, the RNA aptamer SP (SEQ ID NOS: 9 and/or 10) may be incorporated into a system for delivery to cells either in vitro, ex vivo, in situ, or in vivo. In certain aspects, the present disclosure concerns additional RNA sequences to that will assist in transporting RNA aptamers as set forth herein. In certain aspects, the RNA sequences interact to form a junction that will retain the RNA aptamers. In certain aspects, the extra RNA sequences include those of the 3WJ pRNA systems. In some aspects, the 3WJ RNA sequences include those set forth in FIG. 3A and SEQ ID NOS: 12-17:

(SEQ ID NO: 12; 3WJ LEFT)
GUGUAUGUGUACCGG
(SEQ ID NO: 13; 3WJ LEFT REV)
GGCCAUGUGUAUGUG
(SEQ ID NO: 14) 3WJ TOP)
CACAUACUUUGUUGAUCC
(SEQ ID NO: 15 3WJ TOP REV)
CCUAGUUGUUUCAUACAC
(SEQ ID: 16; 3WJ RGT)
CCGGUACUAACUAGG
(SEQ ID NO: 17; 3WJ RGT REV)
GGAUCAAUCAUGGCC.

The 3WJ is derived from the DNA packaging motor of bacteriophage phi29 and has previously shown promise in optimizing targeted delivery of RNA aptamers and other functionally diagnostic and therapeutic moieties (Keefe et al. Nature reviews Drug discovery. 2010; 9(7):537; Shu et al. Methods. 2011; 54(2):204-14; Germer et al. RNA Nanotechnology and Therapeutics CRC Press, Boca Raton, Florida. 2013:399-408; Jasinski et al. ACS nano. 2017; 11(2):1142-64). Notably, previous 3-WJ pRNA nanoparticle delivery systems, with siRNAs, miRNAs, RNA aptamers, fluorescent probes, etc., have demonstrated to provide bio-safe RNA nanoparticles with desirable pharmacokinetic profiles, half-lives and biological activities (Shu et al. Nature protocols. 2013;8(9):1635; Jasinski et al. ACS nano. 2017; 11(2):1142-64; Abdelmawla et al. Molecular Therapy. 2011; 19(7):1312-22; Zhang et al. ACS nano. 2017; 11(1):335-46).

In further aspects, RNA aptamers as set forth herein may be incorporated into delivery systems to allow for or to enhance availability in both in vitro and in vivo systems. In some aspects, the delivery system may include the bacteriophage system identified as phi29. In further aspects, the delivery system may include the phi29 derived pRNA delivery system 3WJ (three way-junction). In certain aspects, the RNA aptamers of the present system may be included with a 3WJ pRNA delivery system.

In some aspects, the RNA aptamers are incorporated having the sequences as set forth in SEQ ID NOS: 12-17. In certain aspects, the present disclosure concerns incorporating any one or more of SEQ ID NOS: 1-11 with the nucleic acids as set forth in SEQ ID NOS: 12-17. It will be appreciated that the 3WJ sequences can include the reverse sequences as well for pairing to form the junction. For example, the sequences of SEQ ID NO: 13, 14 and 17 will form one 3WJ, while the sequences of SEQ ID NOS: 12, 15, and 16 will form a further 3WJ. In certain aspects, the 3WJ of SEQ ID NOS: 13, 14 and 17 are utilized to form the pRNA delivery system.

In some aspects, the present disclosure concerns incorporating a LXXLL (SEQ ID NO: 35) binding RNA aptamer with the 3WJ RNA sequences as set forth in SEQ ID NOS: 12-17. In some aspects, the present disclosure concerns incorporating the P RNA aptamer or a shortened variant thereof (SP) as set forth in SEQ ID NOS: 4, 9 and/or 10 with the 3WJ sequences as set forth in SEQ ID NOS: 12-17.

In some aspects the MED1SP aptamer as set forth in SEQ ID NOS: 9 and 10 are incorporated into the 3WJ-pRNA nanoparticle delivery system.

In some aspects, as described herein, the pRNA nanoparticles are effective and specific for targeting HER2 expressing cells in vitro and in vivo. As is known, HER2 expressing cells are linked to some forms of breast cancers. Breast cancers are divided into different subtypes based on the gene expression profile patterns and cellular markers such as ER and HER2 (Perou et al. Nature. 2000; 406(6797):747-52. Epub 2000/08/30. doi: 10.1038/35021093. PubMed PMID: 10963602). HER2 status of breast cancers often correlates with poorer prognoses and greater risks for metastases and therapeutic resistance (Yu et al. Oncogene. 2000; 19(53):6115). By specific delivery to HER2 expressing cells, the present disclosure provides compositions effective in improving the prognosis of HER2 correlated cancers.

In further aspects, the 3WJ-pRNA system may include a further RNA aptamer, siRNA, or other small RNAs and pharmaceutical agents. In some aspects, the further RNA aptamer may include a HER2-targeting RNA aptamer. In some aspects, the present disclosure includes a MED1SP RNA aptamer as set forth in SEQ ID NOS: 9 and 10 in incorporated in the 3WJ-pRNA delivery system as set forth in SEQ ID NOS: 12-17 and a HER2-targeting RNA aptamer. In some aspects, a HER2-targeting aptamer may include the sequences as set forth in SEQ ID NO: 18: GGGAGGACGAUGCGGUCUGCUGUGCUUGAUAUGCCCCAGACGACUCGCCC (SEQ ID NO: 18; HER2 APTAMER) and SEQ ID NO: 25: GGGAGGACGAUGCGCUCUGCUGUGCUUGAUAUGCCCCAGACGACUCGCCC.

In some aspects, the MED1SP aptamer is incorporated in the 3WJ-pRNA along with a previously characterized HER2-targeting RNA aptamer (Zhang et al. ACS nano. 2017; 11(1):335-46; Thiel et al. Nucleic acids research. 2012; 40(13):6319-37). The resultant pRNA-HER2-MED1SP nanoparticles are capable of successfully entering the nucleus of HER2-expressing breast cancer cells. Importantly, treatment of HER2-expressing breast cancer cells with p-HER2-MED1SP nanoparticles both in vitro and in vivo result in dramatically decreased tumor growth and significantly inhibited metastasis. Importantly, there is no accumulation of the pRNA nanoparticles in other organs, or apparent toxicities or histological abnormalities. Together with the pRNA delivery system, the novel MED1 LXXLL (SEQ ID NO: 35) motif-targeting RNA aptamers as set forth herein provide a therapeutic for metastatic breast cancers.

In further aspects, the pRNA nanoparticles feature an assembly or folding of one or more strands of RNA that include the MED1 aptamers, a HER2 aptamer and a 3WJ. After confirming MED1SPs cellular effects in vitro, the phi29 bacteriophage-derived 3-WJ pRNA nanoparticle system was utilized in order to specifically deliver the MED1SP aptamer to breast cancer cells. The nanoparticle was designed to harbor the MED1SP RNA aptamer in one arm of the 3-WJ pRNA nanoparticle and a HER2-targeting RNA aptamer into the second arm for cancer cell specific targeting (FIG. 3A). The HER2-targeting RNA aptamer was previously characterized for its aptamer-dependent cellular uptake by HER2-expressing breast cancer cells both in vitro and in vivo (Zhang et al. ACS nano. 2017; 11(1):335-46, Thiel et al. Nucleic acids research. 2012; 40(13):6319-37).

Three pieces of RNA were synthesized to form the desired pRNA nanoparticles, having the sequences of each set forth in SEQ ID NOS: 19, 20 and 21 and presented in Table 1 with the 2′ fluoro-abeled bases in lower case letters.

TABLE 1

| SEQ ID NO. | pRNA part | Sequence |
|---|---|---|
| 19 | P1 | GGccAuGuGuAuGuGGG |
| 20 | P2 | GAAGAcAcGAGAGGAuccAcAuuuucGGAucAGuGcGcuuuGAc |
| | | GcAAucuuccccAcAuAcuuuGuuGAucc |
| 21 | P3 | GGGAGGAcGAuGcGcucuGcuGuGcuuGAuAuGccccAGAcGAcu |
| | | cGcccGGAucAAucAuGGcc |

It will be appreciated that P1 (SEQ ID NO: 19) includes SEQ ID NO: 13 of the 3WJ, P2 (SEQ ID NO: 20) includes SEQ ID NOS: 9, 10 and 14 for the MED1 aptamer and the 3WJ and P3 (SEQ ID NO: 21) includes SEQ ID NO: 25 and 17 of the HER2 aptamer and the final 3WJ sequence. The three pieces (P1, P2 and P3) of RNA were mixed in equimolar amounts with annealing buffer to allow for their self-assembly into the control and p-HER2-MED1SP RNA nanoparticles (FIG. 3B). As a control, a pRNA with the previously described HER2-Scramble sequence was used in place of the P2 fragment (SEQ ID NO: 22: GGccAccuccuAGuGcGGAucAGAAcGAAucAGuuuGuucuGAGuAcucGAAAcccAcAuAcuuuGu uGAucc) and annealed with P1 and P3 in equimolar amounts to also self-assemble.

In some aspects, the present disclosure concerns a pRNA nanoparticle comprised of P1, P2 and P3 or SEQ ID NOS; 19-20, herein referred to as p-HER2-MED1SP. It will be appreciated that the pRNA can be assembled as three parts, or divided into further subparts. It will also be appreciated that the pRNA can be assembled through fewer subparts, such as through two parts or a single strand. In some aspects, the pRNA may have a sequence as set forth in SEQ ID NO: 26 (with optional 2′-fluoro (2′F) labels in lower case):

GGccAuGuGuAuGuGGGGAAGAcAcGAGAGGAuccAcAuuuucGGAucA
GuGcGcuuuGAcGcAAucuuccccAcAuAcuuuGuuGAuccGGGAGGAc
GAuGcGcucuGcuGuGcuuGAuAuGccccAGAcGAcucGcccGGAucAA
ucAuGGcc.

It will be appreciated that the present disclosure further encompasses, in some aspects, nucleic acid sequences, including RNA (L- and D-), ssDNA and dsDNA having at least about 70% identity to SEQ ID NOS: 19, 20, 21, and 26, including about 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.9 and 100% identity. In certain aspects, the RNA aptamers may include a RNA sequence of about 85 to about 100% identity with the sequences as set forth in SEQ ID NOS: 19, 20, 21 and 26. In yet other aspects, the present disclosure further encompasses complementary strands and double stranded variants of SEQ ID NOS: 19, 20, 21, and 26, as well as variants that include additional nucleic acids of about 1 to about 15 bases in length, including 2, 3, 4, 5, 6, 7, 8, 9, 10, 11, 12, 13, and 14 nucleic acids on either or both of the termini of the aptamer (5′ and/or 3′). In further aspects, it is to be understood that single-stranded (ss) and double-stranded (ds) deoxyribonucleic acid (DNA) variants are also considered for SEQ ID NOS: 19, 20, 21 and 26. It will be appreciated that as described herein, any replacement or additional base modification to provided nuclease resistance is also contemplated, including application of L-stranded RNA, 3′ O-methyl bases, 2′ fluoro bases, 2-O-methyl bases, 3′ deoxy bases, 3′ ribo bases, phosphorothioate linkages and/or propyne bases. In further aspects, the pRNA may comprise a RNA sequence of from about 85 to about 100% identity to a nucleic acid sequence as set forth in SEQ ID NOS: 19, 20, 21, and/or 26 and wherein the sequence includes about 20 or more consecutive bases as set forth in SEQ ID NOS: 19, 20, 21 and/or 26.

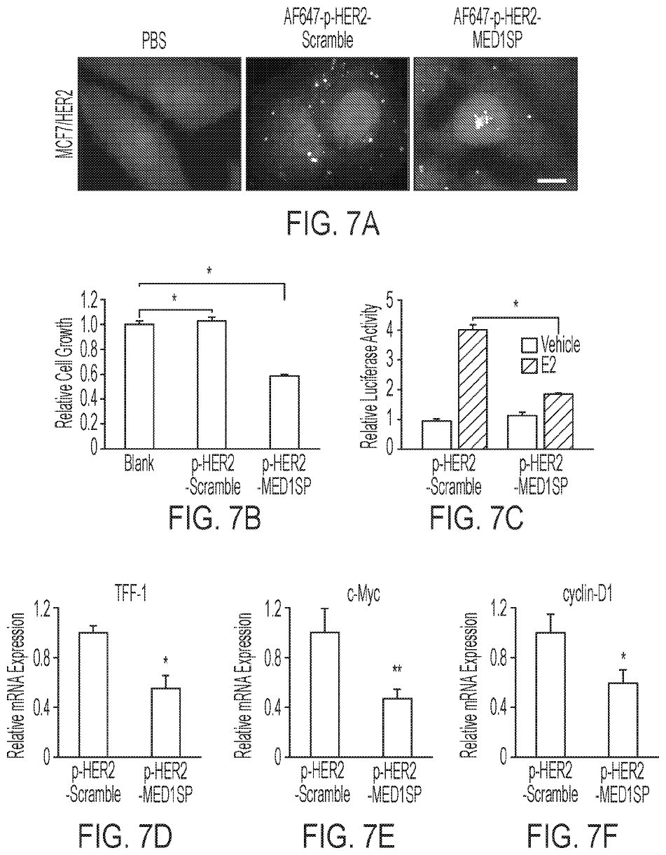

Atomic Force Microscopy (AFM) showed the predicted V-shaped structure (FIG. 3C) of the self-assembled pRNA nanoparticle. To confirm the stability of the 2′F-modified pRNA nanoparticles, p-HER2-Scramble, p-HER2-MED1SP, or unmodified RNA were treated with DMEM+10% FBS for 0, 1, 6, and 24 hours, and it was found that only the unmodified RNA was degraded (FIG. 3D). Likewise, when p-HER2-Scramble, p-HER2-MED1, and unmodified RNA were treated with 0, 1, and 10 μg/mL of RNase A, only unmodified RNA underwent degradation (FIG. 3E). In addition, the ability to enter HER2-expressing breast cancer cells was tested. Alexa647 fluorophore-tagged versions of the above nanoparticles were generated and monitored for their cellular uptake via confocal microscopy. The resulting findings clearly show the uptake of p-HER2-Scramble and p-HER2-MED1SP by both BT-474 cells (FIG. 3F) and MCF-7/HER2 cells (FIG. 7A).

pRNA Nanoparticles Methods In Vitro

In some aspects, the present disclosure concerns administering the pRNA nanoparticles to a cell. In some aspects, the cells can be in vitro. In other aspects, the cell can be in situ, in vivo, or ex vivo.

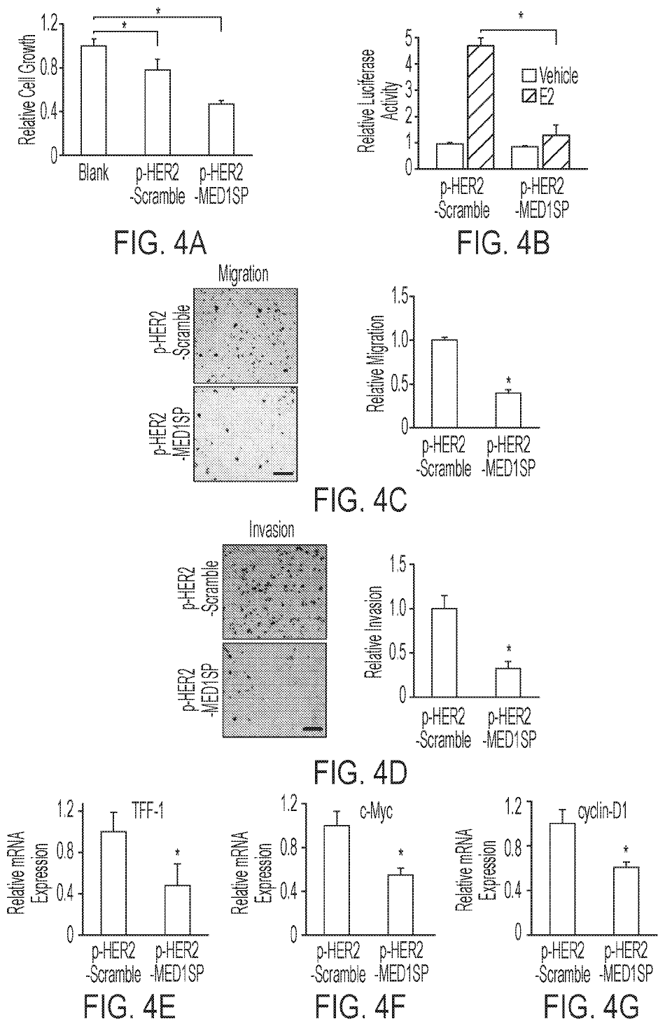

After confirming the cellular uptake of p-HER2-MED1SP nanoparticles, the effects of the pRNA were examined on HER2-expressing breast cancer cell lines in vitro. BT-474 cells are known as HER2 expressing cells. When treated with the pRNA nanoparticles, BT-474 cell growth was significantly decreased by adding p-HER2-MED1SP directly into culture media compared to that of control or no treatment (FIG. 4A). Additionally, p-HER2-MED1SP depleted E2-stimulated ER-reporter gene expression in these cells, as demonstrated by ERE-luciferase assays (FIG. 4B).

Further, the effects of p-HER2-MED1SP and control p-HER2-Scramble treatment on both cell viability and ER-reporter gene expression were confirmed in another HER2-expressing MCF7/HER2 breast cancer cell line (FIG. 7B, 7C).

Because MED1's LXXLL (SEQ ID NO: 35) motifs and their interaction with ER have been shown to play an important role in HER2-mediated breast cancer metastasis, it was further investigated for the effect of the p-HER2-MED1SP nanoparticles on the metastatic capabilities of HER2-expressing breast cancer cells. As shown in FIG. 4C, p-HER2-MED1SP nanoparticle treatment of BT-474 cells greatly reduced the number of cells capable of migrating in a transwell assay, compared to that of p-HER2-Scramble control treated cells. The invasion capabilities of these BT-474 cells was similarly suppressed by p-HER2-MED1SP treatment compared to those treated by controls (FIG. 4D). Moreover, the mRNA expression of ERα-responsive genes involved in cell growth and metastasis was measured using real time PCR analyses and it was found that p-HER2-MED1SP treatment significantly downregulated the expression of these ERα-dependent genes including TFF-1, c-Myc and cyclin D1 (FIG. 4E, F, G).

All of these findings were further confirmed in MCF7/HER2 breast cancer cells as well (FIG. 7B-D). Collectively these results confirm the findings from transfecting MED1SP aptamer into breast cancer cells and demonstrate how the MED1SP aptamer can be incorporated into the compact and stable pRNA delivery scaffold along with a HER2-targeting aptamer to achieve similar functions and efficacies.

pRNA Nanoparticle Methods In Vivo

In other aspects, the pRNA nanoparticle as set forth herein can be administered to a subject, such as to a mammal, including a human. As set forth herein, the MED1 aptamers show specificity for inhibiting MED1 and ER interaction through the LXXLL (SEQ ID NO: 35) motif. While transfection of the aptamers as set forth in SEQ ID NOS: 1-11 is possible, a further vehicle for in vivo delivery may be required to provide for successful cellular uptake. In some aspects, coupling the MED1 aptamers with a HER2 aptamer may provide a mechanism for site-specific cellular delivery to HER2 expressing cells. Further, coupling the MED1 aptamers with a bacteriophage derived 3WJ-pRNA system provides a vehicle to deliver the MED1 aptamer to a cell nucleus and with the HER2 aptamer presence, the MED1 aptamer is delivered to the nucleus of a HER2 expressing cell. Finally, the 2′fluoro labeling of the MED1 aptamer and the overall pRNA provides a protection for the RNA from degradation. Collectively, the pRNA-HER2-MED1 nanoparticles that feature 2′fluoro labeling or similar for nuclease resistance can be delivered successfully to HER2 expressing cells in vivo and therein prevent or inhibit ER activation and/or stimulation by MED1. It will be appreciated that as demonstrated herein, the pRNA nanoparticles did not require a further vehicle in order to successfully enter HER2 expressing cells and enter the nucleus thereof.

In some aspects, the pRNA nanoparticles can be administered alone or with a pharmaceutically acceptable carrier or excipient. Such are set forth in the most recent edition of Remington: The Science and Practice of Pharmacy (23 rd Ed., Academic Press, 2020), which is hereby incorporated by reference in its entirety. In other aspects, the pRNA nanoparticles can be administered to a subject through systemic administration, including intravenous administration, oral administration, subdermal or dermal administration, or sublingual administration. In some instances a further vehicle may be co-administered with the pRNA nanoparticles.

It will also be appreciated that as the pRNA aptamers show efficacy in vivo for HER2 expressing tumors, the administration of such can be coupled with other therapies and therapeutics designed to inhibit or terminate tumor cell growth. In certain aspects, the pRNA nanoparticles may be coupled with one or more of raloxifene, tamoxifen, abemacicib, paclitaxel, everolimus, ado-trastuzumab emtansine, alpelisib, anastrozole, pamidronate, exemestane, atezolizumab, capecitabine, cyclophosphamide, docetaxel, doxorubicin, epirubicin, fluorouracil, fam-trastuzumab deruxtecan-nxki, toremifene, fulvestrant, letrozole, gemcitabine, goserelin, trastuzumab, palbociclib, ixabepilone, ribociclib, lapatinib ditosylate, letrozole, olaparib, megestrol, methotrexate, neratinib, pertuzumab, sacituzumab govitecan-hziy, thiotepa, tucatinib, vinblastine, any salts thereof and combinations thereof.

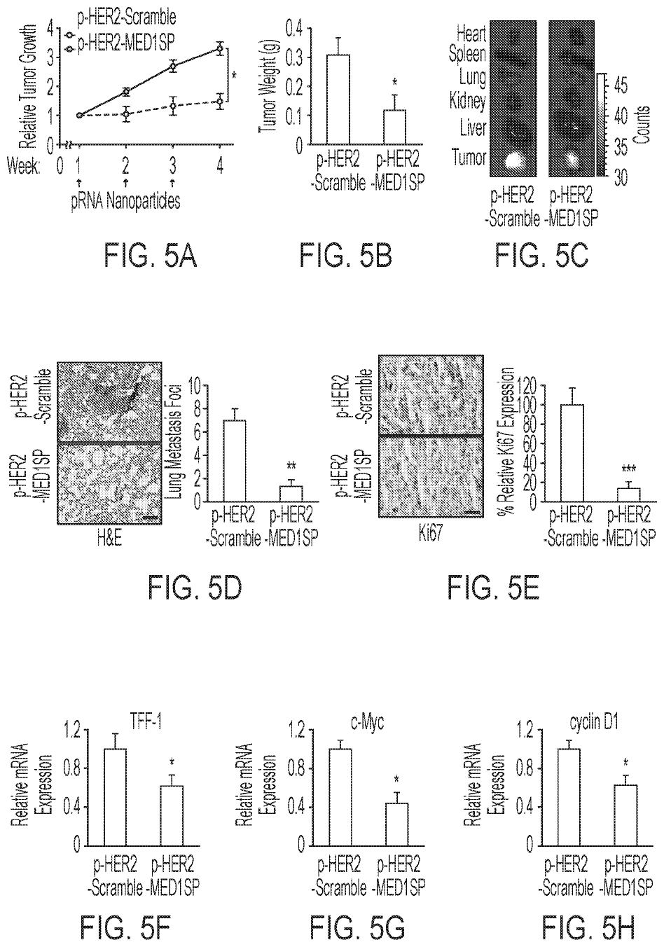

The therapeutic effects of p-HER2-MED1SP nanoparticles in vivo are confirmed herein. As set forth, a BT-474 orthotopic xenograft mouse model was utilized, with the mice randomly designated to either p-HER2-MED1SP or p-HER2-Scramble treatment groups once tumors were of 150 mm 3 or more. The RNA nanoparticles were systemically administered once per week for three weeks. The p-HER2-MED1SP treated mice greatly suppressed tumor growth when compared to tumor growth of control treated mice (FIG. 5A, B). Further, both p-HER2-MED1SP and p-HER2-Scramble nanoparticles localized specifically to HER2-expressing implanted tumors, with no apparent accumulation in other organs such as heart, spleen, lung, kidney or liver (FIG. 5C), confirming the HER2 aptamer functioned for cell specific delivery.

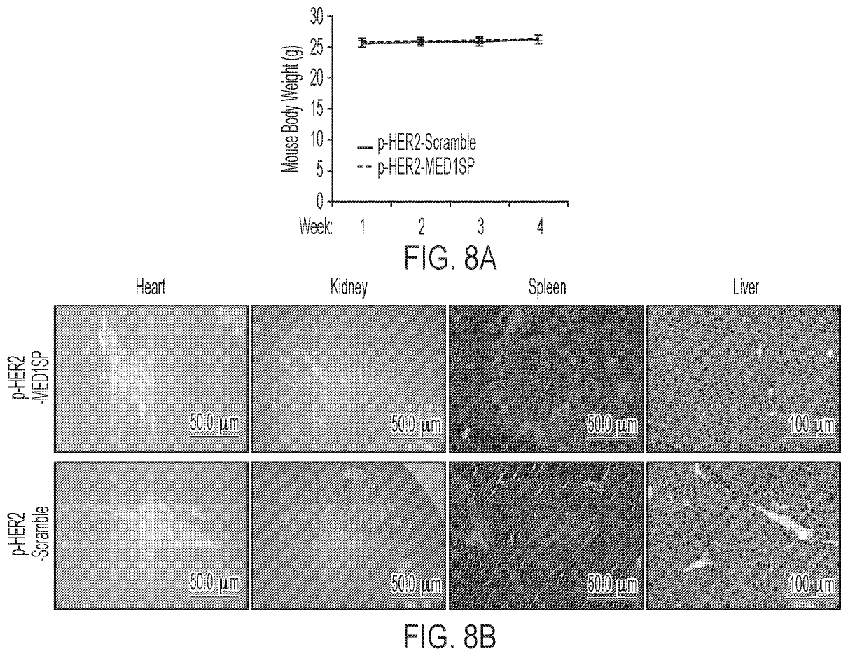

Further, it was identified that not only did the pRNA nanoparticles specifically target HER2-expressing tumors, but also that there was no visible toxicity or apparent side effects associated with nanoparticle treatment (FIG. 8A, B).

With regard to metastasis, it is also demonstrated herein that the pRNA nanoparticle p-HER2-MED1SP greatly reduced the number of metastatic lung nodules compared to those treated with the control p-HER2-Scramble nanoparticles (FIG. 5D).

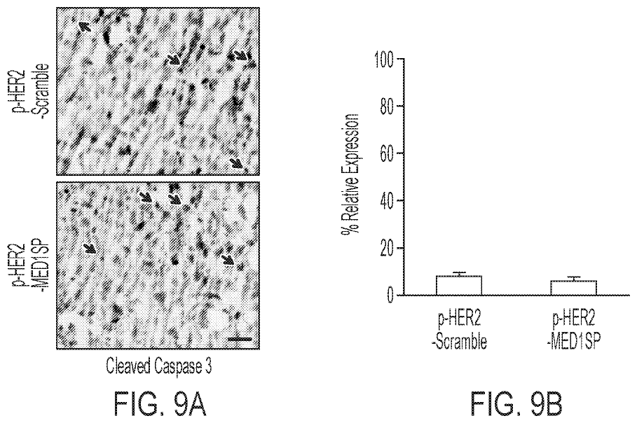

It is also demonstrated herein that the MED1 aptamer affects cell proliferation as opposed to apoptosis in vivo. As set forth herein, p-HER2-MED1SP treatment markedly reduced Ki67 expression when compared to that of p-HER2-Scramble treated mice (FIG. 5E), while the levels of cleaved Caspase-3 were exceptionally low and did not change between treatment groups (FIG. 9A, B). These data are consistent with other studies identifying a role for MED1 in cell proliferation and that the loss in tumor growth was not likely caused by apoptosis.

Lastly, the mRNA levels of the key ER-responsive genes tested above in isolated tumor cells from p-HER2-MED1SP and p-HER2-Scramble treated mice was measured. Consistent with the in vitro studies, p-HER2-MED1SP treated tumor cells expressed significantly lower levels of TFF-1, c-Myc, and cyclin D1 compared to that of p-HER2-Scramble treated mice.

Together, these findings demonstrate the successful systemic delivery of the innovative p-HER2-MED1SP nanoparticles to HER2-expressing tumors, and their deleterious effects on tumor growth, metastasis, and ER-target gene expression. Examples of HER-2 expressing tumors may include breast tumors, bladder tumors, gastric tumors, gallbladder tumors, hepatic tumors, cervical tumors, uterine tumors or testicular tumors.

Collectively, the present invention provides, in some aspects isolated RNA aptamers that affect the PPI between MED1 and an ER. In other aspects, the RNA aptamers are selective for the MED1 LXXLL (SEQ ID NO: 35) motif and do not interact with other LXXLL (SEQ ID NO: 35) motifs. In some aspects, the RNA aptamers target the estrogen receptor-interacting MED1 LXXLL (SEQ ID NO: 35) motifs, a key contributor of HER2-mediated tumorigenesis and metastasis. In further aspects, the present invention provides optimized aptamers for MED1, MED1SP, for delivery and efficacy on HER2-expressing breast cancer cells in both in vitro and in vivo. As demonstrated herein, MED1SP RNA aptamer is highly specific in disrupting ER/MED1 interactions, inhibiting ER-dependent gene expression and disrupting breast cancer cell growth and metastasis. In further aspects, the present disclosure concerns incorporation of MED1SP into a 3-WJ pRNA nanoparticle. In other aspects, the pRNA nanoparticle further harbors a HER2-targeting RNA aptamer. The resulting pRNA-HER2-MED1SP shows tumor specific delivery and effective inhibition of tumor metastasis in vivo without apparent toxicity. In yet further aspects, while the p-HER2-MED1SP nanoparticles are functional for metastatic breast cancer treatments in vivo it will be appreciated that the RNA aptamers can be incorporated with other aptamers and delivery systems for disrupting oncogenic protein-protein interactions and the treatment of other cancers and diseases.

According to a first aspect, either alone or in combination with any other aspect, herein is provided a Mediator Subunit 1 (MED1)-estrogen receptor (ER) binding inhibitor of a ribonucleic acid (RNA) sequence having at least 70% identity to a sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

According to a second aspect, either alone or in combination with any other aspect, the MED1-ER binding inhibitor the nucleic acid sequence has at least 85% identity.

According to a third aspect, either alone or in combination with any other aspect, the MED1-ER binding inhibitor nucleic acid sequence comprises 20 or more consecutive bases to the sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

According to a fourth aspect, either alone or in combination with any other aspect, the MED1-ER binding inhibitor RNA sequence includes SEQ ID NO: 9.

According to a fifth aspect, either alone or in combination with any other aspect, the MED1-ER binding inhibitor RNA sequence includes SEQ ID NO: 10.

According to a sixth aspect, either alone or in combination with any other aspect, one or more nucleic acids of the MED1-ER binding inhibitor includes a modification to be nuclease resistant.

According to a seventh aspect, either alone or in combination with any other aspect, the MED1-ER binding inhibitor may include labelling one or more bases with a fluorine.

According to an eighth aspect, either alone or in combination with any other aspect, all uracil residues of the MED1-ER binding inhibitor may be 2′ fluoro labeled.

According to a ninth aspect, either alone or in combination with any other aspect, all cytosine residues of the MED1-ER binding inhibitor may be 2′ fluoro labeled.

According to a tenth aspect, either alone or in combination with any other aspect, the present disclosure includes pRNA nanoparticle of any RNA aptamer sequence and a 3-way junction (3WJ) nucleotide sequence.

According to an eleventh aspect, either alone or in combination with any other aspect, the 3WJ nucleotide sequence may include SEQ ID NO: 13, SEQ ID NO: 14 and SEQ ID NO: 17.

According to a twelfth aspect, either alone or in combination with any other aspect, a pRNA nanoparticle may include an additional aptamer.

According to a thirteenth aspect, either alone or in combination with any other aspect, a pRNA nanoparticle may include an additional aptamer of a HER2 aptamer.

According to a fourteenth aspect, either alone or in combination with any other aspect, a pRNA nanoparticle may include a HER2 aptamer of a nucleic acid sequence as set forth in SEQ ID NO: 18 or SEQ ID NO: 25.

According to a fifteenth aspect, either alone or in combination with any other aspect, a pRNA nanoparticle may include a nucleic sequence having at least 85% identity to the sequences as set forth in SEQ ID NOS: 19, 20 and 21.

According to a sixteenth aspect, either alone or in combination with any other aspect, a pRNA nanoparticle may include a nucleic acid having at least 85% identity to the sequence as set for in SEQ ID NO: 26.

According to a seventeenth aspect, either alone or in combination with any other aspect, a pRNA nanoparticle may include a modification to one or more nucleic acids to be nuclease resistant.

According to an eighteenth aspect, either alone or in combination with any other aspect, a pRNA nanoparticle a base labelled with a fluorine.

According to a nineteenth aspect, either alone or in combination with any other aspect, a pRNA nanoparticle may include all uracil residues being 2′ fluoro-labeled.

According to a twentieth aspect, either alone or in combination with any other aspect, a pRNA nanoparticle may include all cytosine residues are 2′ fluoro-labeled.

According to a twenty-first aspect, either alone or in combination with any other aspect, the present disclosure includes methods for inhibiting MED1 interacting with an estrogen receptor by administering to a cell a composition comprised of an RNA aptamer, wherein the RNA aptamer includes a ribonucleic acid (RNA) sequence having at least 85% identity to a nucleic acid sequence selected from SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

According to a twenty-second aspect, either alone or in combination with any other aspect, the methods can apply to a cell in vitro.

According to a twenty-third aspect, either alone or in combination with any other aspect, methods can include administering a composition of a pRNA nanoparticle of an RNA aptamer and a 3WJ.

According to a twenty-fourth aspect, either alone or in combination with any other aspect, methods can include cells in vivo.

According to a twenty-fifth aspect, either alone or in combination with any other aspect, methods can include compositions with a pharmaceutically acceptable carrier, a diluent and/or an excipient.

According to a twenty-sixth aspect, either alone or in combination with any other aspect, methods can include cells in a human subject and systemic administration of compounds and/or compositions.

According to a twenty-seventh aspect, either alone or in combination with any other aspect, methods can include treating a HER2 associated tumor in a subject, through systemically administering to the subject with a HER2 associated tumor a composition of a pRNA nanoparticle, wherein the pRNA nanoparticle includes a self-assembled nucleic acid sequence with at least 85% identity to SEQ ID NOS: 19, 20 and 21 or to SEQ ID NO: 26.

According to a twenty-eighth aspect, either alone or in combination with any other aspect, methods can include administration of compositions to a HER2 associated tumor, the HER2 tumor including one or more of a breast tumor, a bladder tumor, a gastric tumor, a gallbladder tumor, a hepatic tumor, a cervical tumor, a uterine tumor or a testicular tumor.

According to a twenty-ninth aspect, either alone or in combination with any other aspect, methods can include compositions of a pRNA nanoparticle and a pharmaceutically acceptable carrier, a diluent and/or an excipient.

According to a thirtieth aspect, either alone or in combination with any other aspect, methods can include administering pRNA nanoparticles systemically.

According to a thirty-first aspect, either alone or in combination with any other aspect, method can include a further chemotherapeutic being administered to the subject.

According to a thirty-second aspect, either alone or in combination with any other aspect, methods can include further chemotherapeutics is selected from raloxifene, tamoxifen, abemacicib, paclitaxel, everolimus, ado-trastuzumab emtansine, alpelisib, anastrozole, pamidronate, exemestane, atezolizumab, capecitabine, cyclophosphamide, docetaxel, doxorubicin, epirubicin, fluorouracil, fam-trastuzumab deruxtecan-nxki, toremifene, fulvestrant, letrozole, gemcitabine, goserelin, trastuzumab, palbociclib, ixabepilone, ribociclib, lapatinib ditosylate, letrozole, olaparib, megestrol, methotrexate, neratinib, pertuzumab, sacituzumab govitecan-hziy, thiotepa, tucatinib, vinblastine, or combinations thereof.

According to a thirty-third aspect, either alone or in combination with any other aspect, the present disclosure includes a MED1-ER binding inhibitor of a ribonucleic acid (RNA) aptamer structure of a single stranded consecutive RNA sequence in the order of: a first stem of 5 base pairs matched with 5 bases of the opposing end of the sequence; 8 unmatched base pairs; a first stem and loop portion comprised of 5 matched pairs and 8 unmatched base pairs; 2 unmatched base pairs; a second stem and loop portion of 4 matched base pairs and 6 unmatched bases; one unmatched base; and the 5 bases of the opposing end of the first stem.

According to a thirty-fourth aspect, either alone or in combination with any other aspect, a pRNA nanoparticle of the RNA aptamer structure and a 3-WJ RNA sequence is included.

Embodiments of inventive compositions and methods are illustrated in the following examples. These examples are provided for illustrative purposes and are not considered limitations on the scope of inventive compositions and methods.

### EXAMPLES

Experimental Methods

Systematic Evolution of Ligands by Exponential Enrichment (SELEX)

A DNA library containing 41nt random sequences flanked by constant sequences (GCACAGCTCGTCTGAATTCTG-N₄₁-GTGGATCCTCTCGTGTCTTCCTATAGTGAGTCGTATTA) were first synthesized (Sigma). RNA transcriptions were then carried out using 2′-fluoro-modified UTPs and CTPs and unmodified ATPs and GTPs at 37° C. overnight using T7 RNA polymerases (40). The resultant RNA was analyzed by 8M urea denaturing 8% PAGE gel, from which it was excised and eluted. This newly purified RNA library was first loaded onto an immobilized MED1 Mutant (LXXAA (SEQ ID NO: 36)) protein column and the flow-through was then incubated with purified MED1 WT (LXXLL (SEQ ID NO: 35)) protein. Bound RNAs were extracted from MED1 WT protein using phenol chloroform (FisherSci) and precipitated. The recovered RNAs were reverse-transcribed by AMV-Reverse Transcriptase into cDNA and further amplified by PCR. Double-stranded DNA (dsDNA) was then transcribed back into an enriched RNA library as described above and used to begin the next SELEX cycle. Six total rounds of SELEX were completed and the recovered RNAs were sequenced after rounds 5 and 6 through cloning/Sanger Sequencing and RNA-seq, respectively, at the CCHMC DNA Sequencing and Genotyping Core (Cincinnati, OH).

Generation and Characterization of pRNA Nanoparticles. pRNA nanoparticles were generated by mixing the equal molar concentrations of p1, p2 and p3 strands (see table 1) synthesized by TriLink and ExonanoRNA, LLC and gradually annealing them from 90 to 20° C. in 1× RNA annealing buffer containing 50 mM Tris—HCl (pH 7.5), 50 mM NaCl, and 1 mM EDTA in a PCR machine and analyzed by 8% native PAGE gels. AF647-conjugated pRNA nanoparticles were generated by incorporating a 5′-terminal AF647-conjugated P1 RNA strand (TriLink) into the annealing process. Atomic Force Microscopy (AFM) imaging of the pRNA nanoparticles was performed at the University of Nebraska Medical Center imaging facility as previously described (Zhang et al. ACS nano. 2017; 11(1):335-46, Shlyakhtenko et al. Ultramicroscopy. 2003;97(1-4):279-87). To test pRNA nanoparticle stability, p-HER2-Scramble, p-HER2-MED1SP or unmodified RNA were exposed to RNase A or 10% FBS supplemented DMEM at the indicated concentrations and durations, respectively, at 37° C. and analyzed by 8% Native PAGE gel electrophoresis.

Cell Culture and pRNA Nanoparticle Cellular Uptake Assay. Human MCF-7, BT474 and MCF-7/HER2 breast cancer cells were cultured in DMEM (Hyclone) supplemented with 10% fetal bovine serum (FBS, Sigma) and 1% penicillin/streptomycin (Corning) at 37° C. in a humidified atmosphere with 5% CO2. For cellular uptake assays, BT474 and MCF-7/HER2 cells were seeded into 8-well chamber slides (Thermo scientific Nunc) and treated with 10 μg/mL of Alexa647-conjugated pRNAs at 37° C. for 12 hours. After washing extensively with PBS, cells were then fixed with 4% paraformaldehyde (FisherSci), permeated with 0.25% Triton X-100 (FisherSci), stained with DAPI (Sigma) and analyzed by Confocal microscopy (Zeiss).

MTT, Migration and Invasion Assays. MCF-7, BT474 and MCF-7/HER2 human breast cancer cells were treated with 10 μg/mL of RNA Aptamers or pRNA nanoparticles for 48 hours. Subsequently, 10 μl of MTT reagent (Sigma) was added to culture medium and incubated at 37° C. for another 4 hours. Resultant formazan crystals were dissolved by perturbation with DMSO, and the 570 nm absorbance of the solution was measured via a microplate reader (BioTek). For migration and invasions assays, cells were transfected with RNA aptamers (via lipofectamine) or directly administered with pRNA nanoparticles in serum-free DMEM (ThermoFisher) media.

Following treatment and starvation, cells were seeded in the upper chamber of a Transwell. The lower chamber was filled with 600 μL DMEM+10% FBS and incubated at 37° C. for 24 hours. For invasion assays, Transwell membrane inserts were first coated with diluted Matrigel (Corning) and incubated briefly prior to cell seeding. Non-migrated/invaded cells were removed by extensive washing. Migrated/invaded cells were fixed to the membrane, stained with 0.1% crystal violet in 20% ethanol and analyzed by an Olympus SX12 microscope.

Orthotopic Xenograft Breast Tumor Mouse Model, in Vivo Imaging and Antitumor Therapy. Five-week old athymic female mice were orthotopically injected with 5×106 BT474 cells mixed 1:1 with matrigel into their fourth mammary gland fat pads. When the tumor sizes reached approximately 150 mm 3, the mice were randomly divided into 2 groups (4/group) and intravenously injected via tail vein with 4 mg/kg of indicated AF647-tagged RNA nanoparticles once a week for 3 weeks. Tumor growth was measured with calipers every 3 days, and tumor volume was calculated using the formula: volume=0.5×(width)2×length. At the end of treatment, mice were sacrificed and organs (heart, spleen, lung, kidneys and liver) and tumors were harvested for further analyses. Tumor weights were recorded and tumor and organ tissues were flash frozen or fixed in 10% formalin for further histological and molecular analyses.

Nanoparticle uptake imaging of harvested tumors and major organs following 3-week treatment was conducted using the IVIS Lumina imaging system with Living Images 3.0 software (Caliper Life Sciences).

Statistics. Data were represented as mean±SEM from at least three independent experiments. The differences among groups were calculated using Student's t-test or one-way ANOVA analysis (GraphPad).

Aptamer-MED1 Binding and GST Pull-Down Assay.

Top MED1 LXXLL (SEQ ID NO: 35) motif-targeting RNA aptamers were incubated with immobilized MED1 WT LXXLL (SEQ ID NO: 35) motif protein or MED1 Mutant LXXAA (SEQ ID NO: 36) motif protein for 2 hours at 4° C. After extensively washing with BC150, the bound RNA was then extracted with phenol chloroform (FisherSci), precipitated, dissolved and analyzed by 8M urea denaturing PAGE gel.

GST pull-down assays were conducted as described previously (Zhang et al. Molecular cell. 2005; 19(1):89-100). Briefly, purified MED1 LXXLL (SEQ ID NO: 35) motif WT proteins or Hela cell nuclear extracts (NE) were mixed with immobilized GST or GST-ER fusion proteins, and the indicated amount of RNA aptamer was added and incubated at 4° C. for 4 hours. After extensive washes with BC200 buffer+E2, bound proteins were boiled for 10 mins in 1× SDS running buffer and analyzed via Western blotting.

Western Blot Analyses Protein samples were denatured in 10X SDS buffer and subjected to SDS-PAGE gel electrophoresis. After being transferred onto a nitrocellulose membrane, immunoblotting was conducted with anti-MED1 (Zhang et al. Molecular cell. 2005; 19(1):89-100), anti-SRC1 and anti-PGC-1B antibodies (Santa Cruz), followed by Horseradish peroxidase-coupled secondary antibody (Jackson ImmunoResearch) treatment and use of an ECL Western Blotting substrate Kit (Pierce) for visualization.

Real-Time PCR

RNeasy Mini Kit (Qiagen) or Trizol reagent (ThermoFisher) were used to extract total RNA from BT-474 and MCF7/HER2 breast cancer cells or orthotopic xenograft tumor tissues, respectively, followed by reverse-transcription with Superscript IV reverse transcriptase (Invitrogen). SYBR Green Master Mix (ThermoFisher) was used to perform Real-time PCR in a 7900HT Fast Real-time system (Applied Biosystems). Expression levels of TFF1 (primers set forth in SEQ ID NOS: 27 and 28), c-Myc (primers set forth in SEQ ID NOS: 29 and 30), and cyclin D1 (primers set forth in SEQ ID NOS: 31 and 32) were analyzed with GAPDH as the internal reference (primers set forth in SEQ ID NOS: 33 and 34).

H&E and Immunohistochemical (INC) Staining. Heart, liver, spleen, lung, kidneys and tumors were harvested from sacrificed mice and fixed in 10% neutral buffered formalin, then dehydrated with a gradient series of ethanol concentrations (70%, 80%, 90%, and 100%). Dehydrated tissues were then embedded in paraffin and cut into 5 μm sections, which were then stained with hematoxylin and eosin (Sigma) and analyzed for histology. To examine the Ki-67 and cleaved Caspase-3 expression of tumors, IHC staining was performed by deparaffinizing tumor tissue sections followed by heat-induced antigen retrieval with citrate buffer and then incubation with anti-Ki-67 or anti-cleaved Caspase-3 primary antibodies (Thermo scientific) overnight at 4° C. Sections were then incubated with a biotin-SP-conjugated secondary antibody (Jackson ImmunoResearch). Vectastain ABC kits and subsequent incubation with DAB substrates (Vector Lab) were used to develop staining, followed by hematoxylin counterstaining of nuclei. Lastly, sections underwent dehydration steps as above, were cleared in Xylene (FisherSci), mounted with Permount (FisherSci) and analyzed with an Axioplan Imaging 2e microscope (Zeiss).

### Results and Discussions

### Isolation and Characterization of MED1 LXXLL (SEQ ID NO: 35) Motif-Targeting RNA Aptamers

The SELEX approach to isolate MED1 LXXLL (SEQ ID NO: 35) motif-targeting RNA aptamers was carried out as shown in FIG. 1A and described in detail in the Experimental Methods. For the starting library design, the constant sequences flanking the randomized region were arranged to prevent hairpin-folding and allow for greater flexibility and diversity. This randomized DNA library was first transcribed to RNA (FIG. 1B), which was then put through a negative selection of purified MED1 mutant (LXXAA (SEQ ID NO: 36)) protein followed by a positive selection of purified MED1 WT (LXXLL (SEQ ID NO: 35)) protein (FIG. 1C). Any RNA species that bound to MED1 WT but not MED1 mutant proteins were then eluted, reverse transcribed and further amplified by PCR into double stranded DNA (dsDNA) (FIG. 1D). The dsDNA was then transcribed into a new, enriched RNA library to initiate a new SELEX cycle. Importantly, the recovery rate of RNA was increased significantly during each round of SELEX (FIG. 1E), and the resultant sequences of the top most enriched RNA aptamers after 6 rounds are shown in FIG. 1F.

For functional characterization of these top 8 RNA aptamer candidates, basic binding assays to compare their binding abilities to MED1 were performed. As shown in FIG. 6A, in contrast to the control scramble (S) aptamer, many of these MED1 RNA aptamers (B, G, P, O, K and X) can bind to MED1 WT but not MED1 mutant proteins. Further, GST pull-down assays demonstrated the ability of aptamer candidates B, G, P, O, K, and X to significantly disrupt the interaction between ER and MED1 (FIG. 6B). Moreover, we measured the ability of each individual aptamer to affect ER-mediated gene expression in ER positive MCF-7 breast cancer cells and the results showed that aptamers R, P, T, O, X were able to strongly inhibit estrogen-dependent ER-reporter gene expression (FIG. 6C). Finally, MTT cell proliferation assays and transwell migration assays were carried out and it was found that aptamers P and T have the most significant effects on growth (FIG. 6D) while aptamers P, T, and O resulted in the greatest loss in migration capabilities (FIG. 6E, 6F). Based on these evidences, RNA aptamer P was selected as the top MED1 aptamer candidate because it consistently demonstrated capabilities in binding MED1 WT, disrupting the ER/MED1 interaction, ER-dependent transcriptional activities, and significant inhibition of both growth and migration of breast cancer cells.

### MED1 RNA Aptamer SP Selectively Disrupts ER/MED1 Interaction, Cell Growth and Metastasis Capabilities of Breast Cancer Cells In Vitro

Upon further analysis of the predicted secondary structure of the P aptamer provided by mFOLD, a loop-stem-loop-stem-loop motif was identified (FIG. 2A, inside box). Deletion-mapping experiments were conducted and it was found that the shortened P (SP) aptamer can block the expression of ER-reporter genes as efficiently as the full-length P aptamer (FIG. 2B). Further, MED1SP can effectively block the ER/MED1 interaction in GST pull-down assays (FIG. 2C).

Interestingly, while the interaction of ER/MED1 is disrupted by MED1SP, it was found that other transcriptional ER co-activators like SRC and PGC-1(3, both of which contain LXXLL (SEQ ID NO: 35) motifs, can still bind ER (FIG. 2D). Moreover, MTT assays confirmed that MED1SP was able to significantly inhibit cell growth (FIG. 2E). Importantly, transfection of MED1SP into BT-474 cells, a HER2-overexpressing metastatic breast cancer cell line, also resulted in a loss in their migration and invasion capabilities, as demonstrated by transwell migration and invasion assays (FIG. 2F, 2G). Overall, these data support a functional selectivity of our MED1SP RNA aptamer and its ability to inhibit ER-mediated transcription and functions.

Construction and Characterization of 3-WJ pRNA-HER2-MED1SP Nanoparticles for HER2-Expressing Breast Cancer Cell Delivery

After confirming MED1SPs cellular effects in vitro, the Phi29 bacteriophage derived 3-WJ pRNA nanoparticle system was utilized in order to specifically deliver the MED1SP aptamer to breast cancer cells. The nanoparticle was designed to harbor the MED1SP RNA aptamer in one arm of the 3-WJ pRNA nanoparticle and a HER2-targeting RNA aptamer into the second arm for cancer cell specific targeting (FIG. 3A). The HER2-targeting RNA aptamer was previously characterized and confirmed for its aptamer-dependent cellular uptake by HER2-expressing breast cancer cells both in vitro and in vivo (Zhang et al. ACS nano. 2017; 11(1):335-46, Thiel et al. Nucleic acids research. 2012; 40(13):6319-37). Three pieces (P1, P2 and P3) of RNA were synthesized and mixed in equimolar amounts with annealing buffer to allow for their self-assembly into the control and p-HER2-MED1SP RNA nanoparticles (FIG. 3B).

Atomic Force Microscopy (AFM) showed the predicted V-shaped structure (FIG. 3C) of the self-assembled nanoparticle. To confirm the stability of the 2′F-modified pRNA nanoparticles, p-HER2-Scramble, p-HER2-MED1SP, or unmodified RNA were treated with DMEM+10% FBS for 0, 1, 6, and 24 hours, and found that only the unmodified RNA was degraded (FIG. 3D).

Likewise, when p-HER2-Scramble, p-HER2-MED1, and unmodified RNA were treated with 0, 1, and 10 μg/mL of RNase A, only unmodified RNA underwent degradation (FIG. 3E).

In addition, their ability to enter HER2-expressing breast cancer cells was tested. Alexa647 fluorophore-tagged versions of the above nanoparticles were generated and monitored their cellular uptake via confocal microscopy. The findings clearly show the uptake of p-HER2-Scramble and p-HER2-MED1SP by both BT-474 cells (FIG. 3F) and MCF-7/HER2 cells (FIG. 7A).

p-HER2-MED1SP Nanoparticles Disrupt Cell Growth, ER-Reporter Gene Expression and Metastasis Capabilities of HER2-Expressing Breast Cancer Cells In Vitro

After confirming the cellular uptake of p-HER2-MED1SP nanoparticles, their effects on HER2-expressing breast cancer cell lines in vitro was examined. It was found that BT-474 cell growth was significantly decreased by adding p-HER2-MED1SP directly into culture media compared to that of control or no treatment (FIG. 4A). Additionally, p-HER2-MED1SP depleted E2-stimulated ER-reporter gene expression in these cells, as demonstrated by ERE-luciferase assays (FIG. 4B). Further, the effects of p-HER2-MED1SP and control p-HER2-Scramble treatment on both cell viability and ER-reporter gene expression were confirmed in another HER2-expressing MCF7/HER2 breast cancer cell line (FIG. 7B, 7C).

Because MED1s LXXLL (SEQ ID NO: 35) motifs and their interaction with ER have been shown to play an important role in HER2-mediated breast cancer metastasis, the effect of the p-HER2-MED1SP nanoparticles on the metastatic capabilities of HER2-expressing breast cancer cells was further investigated. As shown in FIG. 4C, p-HER2-MED1SP nanoparticle treatment of BT-474 cells greatly reduced the number of cells capable of migrating in a transwell assay, compared to that of p-HER2-Scramble control treated cells. The invasion capabilities of these BT-474 cells was similarly suppressed by p-HER2-MED1SP treatment compared to those treated by controls (FIG. 4D). Moreover, the mRNA expression of ERα-responsive genes involved in cell growth and metastasis was measured using real time PCR analyses and it was found that p-HER2-MED1SP treatment significantly downregulated the expression of these ERα-dependent genes including TFF-1, c-Myc and cyclin D1 (FIG. 4E, F, G).

Importantly, all of these findings were confirmed in MCF7/HER2 breast cancer cells as well (FIGS. 7B, 7C and 7D). These results confirm the findings from transfecting MED1SP into breast cancer cells and demonstrate how the MED1SP aptamer can be incorporated into the compact and stable pRNA delivery scaffold along with a HER2-targeting aptamer to achieve similar functions and efficacies.

Inhibition of Breast Cancer Tumor Growth and Metastasis by p-HER2-MED1SP Nanoparticle Treatment In Vivo

To further investigate the therapeutic effects of p-HER2-MED1SP nanoparticles in vivo, a BT-474 orthotopic xenograft mouse model was utilized. Once tumors reached −150 mm 3, mice were randomly designated to either p-HER2-MED1SP or p-HER2-Scramble treatment groups. The RNA nanoparticles were systemically injected via tail vein once per week for three weeks. It was found that p-HER2-MED1SP treated mice greatly suppressed tumor growth when compared to tumor growth of control treated mice (FIG. 5A, B). Importantly, both p-HER2-MED1SP and p-HER2-Scramble nanoparticles localized specifically to HER2-expressing implanted tumors, with no apparent accumulation in other organs such as heart, spleen, lung, kidney or liver (FIG. 5C). Further, it was found that not only did the pRNA nanoparticles specifically target HER2-expressing tumors, but, importantly, there was no visible toxicity or apparent side effects associated with nanoparticle treatment, as the overall body weights and organ morphology were not affected by p-HER2-MED1SP or p-HER2-Scramble treatment (FIG. 8A, 8B).

Next, H&E staining was used to examine how nanoparticle treatment affected lung metastasis and it was found that p-HER2-MED1SP greatly reduced the number of metastatic lung nodules in treated mice compared to those treated with p-HER2-Scramble nanoparticles (FIG. 5D). In addition, to determine whether loss in tumor growth was caused by lower cell proliferation or heightened levels of apoptosis, the levels of Ki67 staining versus the level of cleaved Caspase-3 present was measured in sectioned tumor tissues. It was found that Ki67 expression was reduced by 90% in the p-HER2-MED1SP treated mice when compared to that of p-HER2-Scramble treated mice (FIG. 5E), while the levels of cleaved Caspase-3 were exceptionally low and did not change between treatment groups (FIG. 9A, 9B). This is consistent with previous studies supporting a role for MED1 in cell proliferation and that the loss in tumor growth was not likely caused by apoptosis. Lastly, the mRNA levels of the key ER-responsive genes tested above were measured in isolated tumor cells from p-HER2-MED1SP and p-HER2-Scramble treated mice. Consistent with the in vitro studies, p-HER2-MED1SP treated tumor cells expressed significantly lower levels of TFF-1, c-Myc, and cyclin D1 compared to that of p-HER2-Scramble treated mice.

Together, these findings demonstrate the successful systemic delivery of the innovative p-HER2-MED1SP nanoparticles to HER2-expressing tumors, and their deleterious effects on tumor growth, metastasis, and ER-target gene expression.

Metastatic breast cancer (MBC) is currently incurable and has a devastating estimated 5-year survival rate of less than 30% (Thiel et al. Nucleic acids research. 2012; 40(13):6319-37). While treatments have been developed to improve the prognosis of metastatic breast cancer patients, these are mostly palliative (Reed et al. BMJ supportive & palliative care. 2015; 5(4):358-65). In this study, isolated novel MED1 LXXLL (SEQ ID NO: 35)-motif targeting RNA aptamers were successfully identified through a SELEX approach to disrupt the interaction between ER and its key tissue-specific coactivator MED1.

It has been further confirmed that the MED1SP RNA aptamer can inhibit ER-mediated reporter and endogenous gene expression, breast cancer cell growth and migration and invasion capabilities in vitro. Further, construction and systemic administration of pRNA-HER2-MED1SP RNA nanoparticles in vivo in human breast cancer orthotopic xenograft mouse models demonstrated that these RNA nanoparticles target HER2-expressing tumors specifically and greatly block their growth and lung metastasis without apparent adverse effects.

Throughout decades of basic and clinical oncogenic research, a paramount strategy in stopping the growth and spread of cancers is to target vital signaling pathways (Hanahan. Cell. 2011; 144(5):646-74). Protein-protein interaction (PPI) plays paramount roles in cell signaling and has been considered a highly important and popular target and intervention point (Fry et al. Journal of molecular medicine. 2005; 83(12):955-63, Ivanov et al. Trends in pharmacological sciences. 2013; 34(7):393-400). However, studies found that many protein-protein interactions important for cancer therapy were difficult to be disrupted by small molecules for reasons including large interaction surfaces, unavailable binding pockets, lack of hydrophobic space, difficult interface geometries, etc. (Ivanov et al. Trends in pharmacological sciences. 2013; 34(7):393-400, Emerson et al. Biochemistry. 1995; 34(21):6911-8). Here, isolated RNA aptamers have been successfully identified that are capable of targeting and disrupting an important PPI between the key breast cancer driver ER and its coactivator MED1. Importantly, they have been further confirmed their efficacy in disrupting breast cancer growth and metastasis not only in vitro and but also in vivo in preclinical models. These findings further supported a potential broader use of RNA aptamers for the disruption of other difficult-to-target key PPIs (e.g. Ras/Raf, CDK4/pRB, E3 ubiquitin ligases complex) for the treatment of cancers and other diseases (Ivanov et al. Trends in pharmacological sciences. 2013; 34(7):393-400, Emerson et al. Biochemistry. 1995; 34(21):6911-8, Savkur et al. J Pept Res. 2004; 63(3):207-12. PubMed PMID: 15049832, Fry et al. Journal of molecular medicine. 2005; 83(12):955-63).

Although LXXLL (SEQ ID NO: 35) motifs are present in a variety of ER coactivators, it is also known that the coactivator-specific sequences flanking the LXXLL (SEQ ID NO: 35) motif are important for their interactions with ER (Coulthard et al. Journal of Biological Chemistry. 2003; 278(13):10942-51, Emerson et al. Biochemistry. 1995; 34(21):6911-8). This has led to the concept of potentially targeting LXXLL (SEQ ID NO: 35) motifs to inhibit particular ER-coactivator interactions for the tissue- and gene-specific disruption of ER signaling (Leonard et al. Journal of Zhejiang University-SCIENCE B. 2019; 20(5):381-90, Jiang et al. PNAS. 2010; 107(15):6765-70). Consistent with that, it has been reported that a mutation to the ER AF-2 domain resulted in a selective loss of ER interaction with SRC coactivator, but of that not with MED1 (Acevedo et al. Molecular cell. 2004; 13(5):725-38). The findings from this study further supported a relative specificity of the MED1SP aptamer as it was able to block ER interactions with coactivator MED1 but not with other well-known ER-coactivators such as SRC and PGC-1(3. Importantly, previous research has shown that MED1 LXXLL (SEQ ID NO: 35) motifs plays a rather tissue-, cell- and gene specific role in vivo in regulating ER-mediated functions in mammary gland but not in other estrogen responsive tissues (Jiang et al. PNAS. 2010; 107(15):6765-70).

These, combined with the use of tumor-targeting pRNA nanoparticles that have been shown to have excellent biosafety and pharmacodynamic properties with little toxicity in a growing number of disease models (Jasinski et al. ACS nano. 2017; 11(2):1142-64), are thus likely to provide advantageous therapeutic regimens for patients with minimized side effects. The results are consistent with this perspective as well and no apparent toxicity, body weight change or altered histology of vital organs has been observed following systemic treatment with p-HER2-MED1SP nanoparticles.

Breast cancers are divided into different subtypes based on the gene expression profile patterns and cellular markers such as ER and HER2 (Perou et al. Nature. 2000; 406(6797):747-52. Epub 2000/08/30. doi: 10.1038/35021093. PubMed PMID: 10963602). HER2 status of breast cancers often correlates with poorer prognoses and greater risks for metastases and therapeutic resistance (Yu et al. Oncogene. 2000; 19(53):6115). Importantly, ER/HER2 double positive luminal B breast cancer subtypes are especially difficult to treat because they are highly metastatic and resistant to current therapies. Therefore, it is significant that the p-HER2-MED1SP nanoparticles exhibited great efficacy in inhibiting ER+/HER2+ breast cancer metastasis both in vitro and in vivo in orthotopic xenograft models.

Notably, HER2 is overexpressed in a variety of other cancer types, including lung, gastric, esophageal, ovarian and endometrial cancers (Iqbal et al. Molecular biology international. 2014; 2014). Further, ER signaling that is driven by both hormone expression and HER2-activation has already been shown to play important roles in a number of these cancers (Thomas et al. Nat Rev Cancer. 2011; 11(8):597-608. Epub 2011/07/23. doi: 10.1038/nrc3093. PubMed PMID: 21779010, Verri et al. Oncology. 2005; 68(2-3):154-61), which suggested that the p-HER2-MED1SP nanoparticles therefore could have potential uses beyond breast cancer. In addition, for those ER-driven cancers that do not express HER2, the HER2 aptamer could be replaced with other targeting aptamers (e.g. EpCAM, EGFR, CD44) or moieties (e.g. folate) to facilitate targeted delivery of the nanoparticles and further broaden the use of the MED1SP aptamer (Jasinski et al. ACS nano. 2017; 11(2):1142-64). With recent increases in the FDA approval of RNA based therapeutics, these findings provide a novel approach and highly promising regimen that could make important clinical impact in the treatment of metastatic breast cancers and other diseases.

### CONCLUSIONS

In summary, RNA aptamers have been successfully isolated using the SELEX approach to target the estrogen receptor-interacting MED1 LXXLL (SEQ ID NO: 35) motifs, a key contributor of HER2-mediated tumorigenesis and metastasis. One such aptamer, MED1SP has been further modified and optimized for its delivery and therapeutic effects on HER2-expressing breast cancer cells in both in vitro and in vivo preclinical models. The results showed that the MED1SP RNA aptamer was highly specific in disrupting ER/MED1 interactions, inhibiting ER-dependent gene expression and disrupting breast cancer cell growth and metastasis. Incorporation of MED1SP into a 3-WJ pRNA nanoparticle harboring a HER2-targeting RNA aptamer further demonstrated its tumor specific delivery and effective inhibition of tumor metastasis in vivo without apparent toxicity.

Overall, these studies not only support the use of the novel p-HER2-MED1SP nanoparticles for metastatic breast cancer treatment but could also have broader impacts on the use of RNA aptamers for disrupting oncogenic protein-protein interactions and the treatment of other cancers and diseases.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A Mediator Subunit 1 (MED1)-estrogen receptor (ER) binding inhibitor comprising a ribonucleic acid (RNA) sequence comprised of a nucleic acid sequence with at least 70% identity to a sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

2. The MED1-ER binding inhibitor of claim 1, wherein the nucleic acid sequence has at least 85% identity to the selected sequence.

3. The MED1-ER binding inhibitor of claim 1, wherein the nucleic acid sequence comprises 20 or more consecutive bases to the sequence selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

4. The MED1-ER binding inhibitor of claim 1, wherein the RNA sequence comprises SEQ ID NO: 9.

5. The MED1-ER binding inhibitor of claim 1, wherein the RNA sequence comprises SEQ ID NO: 10.

6. The MED1-ER binding inhibitor of claim 1, wherein one or more cytosine and/or uracil residues is labelled with a fluorine to confer nuclease resistance.

7. (canceled)

8. The MED1-ER binding inhibitor of claim 6, wherein all uracil residues are 2′ fluoro labeled.

9. The MED1-ER binding inhibitor of claim 6, wherein all cytosine residues are 2′ fluoro labeled.

10. A pRNA nanoparticle comprising the MED1-ER binding inhibitor of claim 1 and a 3-way junction (3WJ) nucleotide sequence.

11. The pRNA nanoparticle of claim 10, wherein the 3WJ nucleotide sequence comprises SEQ ID NO: 13, SEQ ID NO: 14 and SEQ ID NO: 15.

12. The pRNA nanoparticle of claim 10, further comprising an additional aptamer.

13. The pRNA nanoparticle of claim 12, wherein the additional aptamer is a HER2 aptamer.

14. The pRNA nanoparticle of claim 13, wherein the HER2 aptamer comprises a nucleic acid sequence as set forth in SEQ ID NO: 18 or SEQ ID NO: 25.

15. The pRNA nanoparticle of claim 10, wherein the pRNA comprises nucleic sequences with at least 85% identity to the sequences as set forth in SEQ ID NOS: 19, 20 and 21.

16. The pRNA nanoparticle of claim 10, wherein the pRNA comprises a nucleic acid with at least 85% identity to the sequence as set for in SEQ ID NO: 26.

17-20. (canceled)

21. A method for inhibiting MED1 interacting with an estrogen receptor comprising administering to a cell the MED1-ER binding inhibitor of claim 1.

22. (canceled)

23. The method of claim 21, wherein the composition is a pRNA nanoparticle comprised of the RNA aptamer and a 3WJ.

24. The method of claim 23, wherein the cell is in vivo.

25. The method of claim 24, wherein the composition further comprises a pharmaceutically acceptable carrier, a diluent or an excipient, and the composition is administered systemically.

26. (canceled)

27. A method for treating a HER2 associated tumor in a subject, comprising systemically administering to a subject with a HER2 associated tumor a composition comprised of a pRNA nanoparticle, wherein the pRNA nanoparticle is comprised of self-assembled nucleic acid sequence with at least 85% identity to SEQ ID NOS: 19, 20 and 21 or to SEQ ID NO: 26 and further wherein the HER2 associated tumor is a breast tumor, a bladder tumor, a gastric tumor, a gallbladder tumor, a hepatic tumor, a cervical tumor, a uterine tumor or a testicular tumor.

28-34. (canceled)


SEQUENCE LISTING (36 sequences)

SEQ ID NO: 1 - RNA, 39 nt, Artificial Sequence
  Synthetic Construct
gcgauggguaaucaacugcaucucccguacagguuacca

SEQ ID NO: 2 - RNA, 40 nt, Artificial Sequence
  Synthetic Construct
cggaagugagagaccaggucaacgcccaaugccaguaucu

SEQ ID NO: 3 - RNA, 40 nt, Artificial Sequence
  Synthetic Construct
cggaaaggcgagaguguucaaagaaccagcaguccacaau

SEQ ID NO: 4 - RNA, 40 nt, Artificial Sequence
  Synthetic Construct
cauuuucggaucagugcgcuuugacgcaaucuuccacaac

SEQ ID NO: 5 - RNA, 40 nt, Artificial Sequence
  Synthetic Construct
cauuuucggaucaggggcuuugccgaguguccuccuacga

SEQ ID NO: 6 - RNA, 39 nt, Artificial Sequence
  Synthetic Construct
cuuuucggauggagaugcuuugucauuugugucuugcac

SEQ ID NO: 7 - RNA, 40 nt, Artificial Sequence
  Synthetic Construct
cuuuucgggucccguuuaaccuuguuaaccgucuuccugc

SEQ ID NO: 8 - RNA, 40 nt, Artificial Sequence
  Synthetic Construct
ugacaugugacuggaguucagacgugugcucuuccgaucu

SEQ ID NO: 9 - RNA, 34 nt, Artificial Sequence
  Synthetic Construct
cauuuucggaucagugcgcuuugacgcaaucuuc

SEQ ID NO: 10 - RNA, 53 nt, Artificial Sequence
  Synthetic Construct
gaagacacgagaggauccacauuuucggaucagugcgcuuugacgcaaucuuc

SEQ ID NO: 11 - RNA, 81 nt, Artificial Sequence
  Synthetic Construct
gaagacacgagaggauccacauuuucggaucagugcgcuuugacgcaaucuucgacaaccgcugaauucagacgagugagc

SEQ ID NO: 12 - RNA, 15 nt, Artificial Sequence
  Synthetic Construct
guguauguguaccgg

SEQ ID NO: 13 - RNA, 15 nt, Artificial Sequence
  Synthetic Construct
ggccauguguaugug

SEQ ID NO: 14 - RNA, 18 nt, Artificial Sequence
  Synthetic Construct
cacauacuuuguugaucc

SEQ ID NO: 15 - RNA, 18 nt, Artificial Sequence
  Synthetic Construct
ccuaguuguuucauacac

SEQ ID NO: 16 - RNA, 15 nt, Artificial Sequence
  Synthetic Construct
ccgguacuaacuagg

SEQ ID NO: 17 - RNA, 15 nt, Artificial Sequence
  Synthetic Construct
ggaucaaucauggcc

SEQ ID NO: 18 - RNA, 50 nt, Artificial Sequence
  Synthetic Construct
gggaggacgaugcggucugcugugcuugauaugccccagacgacucgccc

SEQ ID NO: 19 - RNA, 17 nt, Artificial Sequence
  Synthetic Construct
ggccauguguauguggg

SEQ ID NO: 20 - RNA, 73 nt, Artificial Sequence
  Synthetic Construct
gaagacacgagaggauccacauuuucggaucagugcgcuuugacgcaaucuuccccacauacuuuguugaucc

SEQ ID NO: 21 - RNA, 65 nt, Artificial Sequence
  Synthetic Construct
gggaggacgaugcgcucugcugugcuugauaugccccagacgacucgcccggaucaaucauggcc

SEQ ID NO: 22 - RNA, 73 nt, Artificial Sequence
  Synthetic Construct
ggccaccuccuagugcggaucagaacgaaucaguuuguucugaguacucgaaacccacauacuuuguugaucc

SEQ ID NO: 23 - DNA, 21 bp, Artificial Sequence
  Synthetic Construct
gcacagctcgtctgaattctg

SEQ ID NO: 24 - DNA, 38 bp, Artificial Sequence
  Synthetic Construct
gtggatcctctcgtgtcttcctatagtgagtcgtatta

SEQ ID NO: 25 - RNA, 50 nt, Artificial Sequence
  Synthetic Construct
gggaggacgaugcgcucugcugugcuugauaugccccagacgacucgccc

SEQ ID NO: 26 - RNA, 155 nt, Artificial Sequence
  Synthetic Construct
ggccauguguauguggggaagacacgagaggauccacauuuucggaucagugcgcuuugacgcaaucuuccccacauacuuuguugauccgggaggacgaugcgcucugcugugcuugauaugccccagacgacucgcccggaucaaucauggcc

SEQ ID NO: 27 - DNA, 18 bp, Artificial Sequence
  Synthetic Construct
tcccagtgtgcaaataag

SEQ ID NO: 28 - DNA, 19 bp, Artificial Sequence
  Synthetic Construct
attcacactcctcttctgg

SEQ ID NO: 29 - DNA, 20 bp, Artificial Sequence
  Synthetic Construct
gaagaaattcgagctgctgc

SEQ ID NO: 30 - DNA, 21 bp, Artificial Sequence
  Synthetic Construct
cacatacagtcctggatgatg

SEQ ID NO: 31 - DNA, 16 bp, Artificial Sequence
  Synthetic Construct
cgccccacccctccag

SEQ ID NO: 32 - DNA, 19 bp, Artificial Sequence
  Synthetic Construct
ccgcccagaccctcagact

SEQ ID NO: 33 - DNA, 23 bp, Artificial Sequence
  Synthetic Construct
cggagtcaacggatttggtcgta

SEQ ID NO: 34 - DNA, 24 bp, Artificial Sequence
  Synthetic Construct
agccttctccatggtggtgaagac

SEQ ID NO: 35 - protein, 5 aa, Artificial Sequence
  Synthetic Construct
  misc_feature (2)..(3): Xaa can be any naturally occurring amino acid
LXXLL

SEQ ID NO: 36 - protein, 5 aa, Artificial Sequence
  Synthetic Construct
  misc_feature (2)..(3): Xaa can be any naturally occurring amino acid
LXXAA